(19) United States
(12) Patent Application Publication
(10) Pub. No.: US 2008/0233573 A1
(43) Pub. Date: Sep. 25, 2008

(54) Gene expression profiling for identification, monitoring and treatment of transplant rejection

(76) Inventors: Kathleen Storm, Longmont, CO (US); Danute Bankaitis-Davis, Longmont, CO (US); Lisa Siconolfi, Westminster, CO (US); John Cheronis, Conifer, CO (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/897,160

(22) Filed: Aug. 28, 2007

Related U.S. Application Data
(60) Provisional application No. 60/840,777, filed on Aug. 28, 2006.

(51) Int. Cl. C12Q 1/68

(52) U.S. Cl. 435/6

(57) ABSTRACT

The present invention provides methods of characterizing organ transplant rejection or inflammatory conditions associated with organ transplant rejection using gene expression profiling.

### REFERENCE TO RELATED APPLICATIONS

This non-provisional patent application claims priority under 35 U.S.C. § 119(e) to U.S. Provisional Patent Application Ser. No. 60/840,777, filed Aug. 28, 2006, the contents of which are hereby incorporated by reference in its entirety.

### FIELD OF THE INVENTION

The present invention relates generally to the identification of biological markers associated with immunosuppression. More specifically, the invention relates to the use of gene expression data in the identification, monitoring and treatment of transplant rejection, autoimmune diseases and in the characterization and evaluation of inflammatory conditions induced or related to transplant rejection and autoimmune diseases.

### BACKGROUND OF THE INVENTION

Acute rejection is a major cause of morbidity and mortality in the first 6 months post organ, e.g., lung, kidney, liver, heart or pancreas transplantation. Frequently, by the time symptoms or other clinical findings manifest, significant organ damage has developed and returning the patient to a more stable condition requires aggressive intervention that has its own untoward consequences. In order to detect and treat acute rejection before significant organ dysfunction occurs, lung transplantation programs have increasingly adopted surveillance broncoscopies and transbronchial biopsies, which also carry with them significant clinical risks as well as financial costs. A sensitive, specific, reliable and non-invasive method for identifying patients who will develop acute organ rejection pre-symptomatically would be welcomed by physicians and patients alike.

### SUMMARY OF THE INVENTION

The invention is based in part upon the identification of gene expression profiles (Precision Profiles™) associated with transplant rejection (TX) and immunosuppression. Theses genes are referred to herein as TX-associated genes or TX-associated constituents. More specifically, the invention is based upon the surprising discovery that detection of as few as two TX-associated genes is capable of identifying individuals with or without TX with at least 75% accuracy.

In various aspects the invention provides a method for determining a profile data set for characterizing a subject with transplant rejection, an inflammatory condition related to transplant rejection or immunosuppression based on a sample from the subject, the sample providing a source of RNAs, by using amplification for measuring the amount of RNA in a panel of constituents including at least 1 constituents from any of Tables 1, 2, 3, 4, 5, or 6, and arriving at a measure of each constituent. The profile data set contains the measure of each constituent of the panel. In addition, the invention is based upon the discovery that the methods provided by the invention are capable of detecting transplant rejection or inflammatory conditions related to transplant rejection by assaying blood samples.

Also provided by the invention is a method of characterizing a subject with transplant rejection, an inflammatory condition related to transplant rejection, or immunosuppression, based on a sample from the subject, the sample providing a source of RNAs, by assessing a profile data set of a plurality of members, each member being a quantitative measure of the amount of a distinct RNA constituent in a panel of constituents selected so that measurement of the constituents enables characterization of the presumptive signs of transplant rejection or immunosuppression.

In yet another aspect the invention provides a method of characterizing a transplant rejection, an inflammatory condition related to transplant rejection, or immunosuppression in a subject, based on a sample from the subject, the sample providing a source of RNAs, by determining a quantitative measure of the amount of at least one constituent from Tables 1-6.

The panel of constituents are selected so as to distinguish from a normal and transplant recipient or an immunosuppressed subject, e.g. a medically immunosuppressed subject.

Preferably, the panel of constituents are selected so as to distinguish e.g., classify between a normal and a transplant recipient or an immunosuppressed subject with at least 75%, 80%, 85%, 90%, 95%, 97%, 98%, 99% or greater accuracy. By “accuracy” is meant that the method has the ability to distinguish e.g., classify between subjects having transplant rejection, an inflammatory condition related to transplant rejection, or immunosuppression, and those that do not. Accuracy is determined for example by comparing the results of the Gene Precision Profiling to standard accepted clinical methods of diagnosing transplant rejection, an inflammatory condition related to transplant rejection, or immunosuppression

Alternatively, the panel of constituents is selected as to permit characterizing severity of transplant rejection, an inflammatory condition related to transplant rejection, or immunosuppression in relation to normal over time so as to track movement toward normal as a result of successful therapy and away from normal in response to transplant rejection. Thus, in some embodiments, the methods of the invention are used to determine efficacy of treatment of a particular subject.

The panel contains 10, 8, 5, 4, 3 or fewer constituents. Optimally, the panel of constituents includes TOSO, ICOS, IL32 or LTA, CD69 or IL1R1. The panel includes two or more constituents from any of Tables 1-6.

Optionally, assessing may further include comparing the profile data set to a baseline profile data set for the panel. The baseline profile data set is related to the transplant rejection, an inflammatory condition related to transplant rejection, or immunosuppression to be characterized. The baseline profile data set is derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects. In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess transplant rejection, an inflammatory condition related to transplant rejection, or immunosuppression of the one or more different subjects, and may also include interpreting the calibrated profile data set in the context of at least one other clinical indicator, wherein the at least one other clinical indicator includes blood chemistry, X-ray or other radiological or metabolic imaging technique, other chemical assays, and physical findings.

The baseline profile data set may be derived from one or more other samples from the same subject taken under circumstances different from those of the first sample, and the circumstances may be selected from the group consisting of (i) the time at which the first sample is taken (e.g., before, after, or during treatment for transplant rejection), (ii) the site from which the first sample is taken, (iii) the biological condition of the subject when the first sample is taken.

Also provided by the invention is a method for predicting response to therapy (e.g., individuals who will respond to a particular therapy (“responders), individuals who won't respond to a particular therapy (“non-responders”), and/or individuals in which toxicity of a particular therapeutic may be an issue), in a subject having transplant rejection, an inflammatory condition related to transplant rejection, or immunosuppression, based on a sample from the subject, the sample providing a source of RNAs, the method comprising: i) determining a quantitative measure of the amount of at least one constituent of any panel of constituents in Tables 1-6 as a distinct RNA constituent, wherein such measure is obtained under measurement conditions that are substantially repeatable to produce a patient data set; and ii) comparing the patient data set to a baseline profile data set, wherein the baseline profile data set is related to the transplant rejection, inflammatory condition related to transplant rejection, or immunosuppression. Optimally, the panel of constituents includes TOSO, ICOS, IL32 or LTA, CD69 or IL1R1.

Additionally, the invention includes a biomarker for predicting individual response to transplant rejection treatment in a subject having transplant rejection, inflammatory condition related to transplant rejection, or immunosuppression, comprising at least one constituent of any constituent of Tables 1-6. Optimally, the panel of constituents includes TOSO, ICOS, IL32 or LTA, CD69 or IL1R1.

Also provided by the invention is a method for monitoring the progression of transplant rejection, an inflammatory condition related to transplant rejection, or immunosuppression, based on a sample from the subject, the sample providing a source of RNAs, the method comprising: a) determining a quantitative measure of the amount of at least one constituent of any constituent of Tables 1-6, as a distinct RNA constituent in a sample obtained at a first period of time, wherein such measure is obtained under measurement conditions that are substantially repeatable to produce a first patient data set; b) determining a quantitative measure of the amount of at least one constituent of any constituent of Tables 1-6 as a distinct RNA constituent in a sample obtained at a second period of time, wherein such measure is obtained under measurement conditions that are substantially repeatable to produce a second profile data set; and c) comparing the first profile data set and the second profile data set to a baseline profile data set, wherein the baseline profile data set is related to transplant rejection, an inflammatory condition related to transplant rejection, or immunosuppression.

Also provided is a method of assessing the efficacy of a compound to suppress the immune system in a subject, based on a sample from the subject, the sample providing a source of RNAs, the method comprising: contacting a first sample from said subject with a test compound and determining a first quantitative measure of the amount of at least one constituent from any of Tables 1-6 in said first sample as a distinct RNA constituent to produce a test data set, wherein such measure is obtained under measurement conditions that are substantially repeatable; and comparing the test data set to a baseline data set. In one embodiment, the baseline data set is derived from a second sample from said subject. In another embodiment, the second sample has not been exposed to said test compound.

In another embodiment, the method of assessing the efficacy of a compound to suppress the immune system in a subject, based on a sample from the subject, the sample providing a source of RNAs, comprises: determining a first quantitative measure of the amount of at least one constituent from any of Tables 1-6 in said first sample from said subject that has been exposed to said test compound as a distinct RNA constituent to produce a test data set, wherein such measure is obtained under measurement conditions that are substantially repeatable; and comparing the test data set to a baseline data set. In some embodiments, the baseline data set is derived from a second sample from said subject. In some embodiments, the second sample has not been exposed to said test compound. In some embodiments, the second sample is obtained from said subject prior to exposure to said test compound, whereas in other embodiments, the second sample is obtained from said subject after exposure to said test compound

The sample is any sample derived from a subject which contains RNA. For example the sample is blood, a blood fraction, bodily fluid, and a population of cells or tissue from the subject.

Optionally one or more other samples can be taken over an interval of time that is at least one month between the first sample and the one or more other samples, or taken over an interval of time that is at least 1 week between the first sample and the one or more samples, or they may be taken pre-therapy intervention or post-therapy intervention. In such embodiments, the first sample may be derived from blood and the baseline profile data set may be derived from tissue or bodily fluid of the subject other than blood. Alternatively, the first sample is derived from tissue or bodily fluid of the subject and the baseline profile data set is derived from blood.

All of the forgoing embodiments are carried out wherein the measurement conditions are substantially repeatable, particularly within a degree of repeatability of better than five percent or more particularly within a degree of repeatability of better than three percent, and/or wherein efficiencies of amplification for all constituents are substantially similar, more particularly wherein the efficiency of amplification is within two percent, and still more particularly wherein the efficiency of amplification for all constituents is less than one percent.

Additionally the invention includes storing the profile data set in a digital storage medium. Optionally, storing the profile data set includes storing it as a record in a database.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a plot showing discrimination between normals (N) and lung transplant Subjects L0=nonrejectors, L1=rejectors) provided by the 2 Genes TOSO and CD69 Includes measurements on lung transplant subjects at both week 4 and week 6.

FIG. 2 is a plot showing discrimination between normals (N) and lung transplant Subjects L0=nonrejectors, L1=rejectors) provided by the 2 Genes TOSO and CD69. Includes measurements on lung transplant subjects at week 4.95% of Lung Transplants were correctly classified, 100% of Normals were correctly classified in this two gene model.

FIG. 3 is a plot showing discrimination between normals (N) and lung transplant Subjects L0=nonrejectors, L1=rejectors) provided by the 2 Genes TOSO and CD69. Includes measurements on lung transplant subjects at week 6.95% of Lung Transplants were correctly classified, 100% of Normals were correctly classified in this two gene model.

FIG. 4 is a plot showing discrimination between normals (N) and lung transplant Subjects L0=nonrejectors, L1=rejectors) provided by the 2 Genes ICOS and CD69 Includes measurements on lung transplant subjects at both week 4 and week 6.

FIG. 5 is a plot showing discrimination between normals (N) and lung transplant Subjects L0=nonrejectors, L1=rejectors) provided by the 2 Genes ICOS and CD69. Includes measurements on lung transplant subjects at week 4. 100% of Lung Transplants were correctly classified, 93.3% of Normals were correctly classified in this two gene model.

FIG. 6 is a plot showing discrimination between normals (N) and lung transplant Subjects L0=nonrejectors, L1=rejectors) provided by the 2 Genes ICOS and CD69. Includes measurements on lung transplant subjects at week 6. 100% of Lung Transplants were correctly classified, 93.8% of Normals were correctly classified in this two gene model.

FIG. 7 is a plot showing discrimination between normals (N) and lung transplant Subjects L0=nonrejectors, L1=rejectors) provided by the 2 Genes IL32 and CD69 Includes measurements on lung transplant subjects at both week 4 and week 6.

FIG. 8 is a plot showing discrimination between normals (N) and lung transplant Subjects L0=nonrejectors, L1=rejectors) provided by the 2 Genes IL32 and CD69. Includes measurements on lung transplant subjects at week 4.95% of Lung Transplants were correctly classified, 93.8% of Normals were correctly classified in this two gene model.

FIG. 9 is a plot showing discrimination between normals (N) and lung transplant Subjects L0=nonrejectors, L1=rejectors) provided by the 2 Genes IL32 and CD69. Includes measurements on lung transplant subjects at week 6. 100% of Lung Transplants were correctly classified, 93.8% of Normals were correctly classified in this two gene model.

FIG. 10 is a plot showing discrimination between normals (N) and lung transplant Subjects L0=nonrejectors, L1=rejectors) provided by the 2 Genes TNFRSF5 and ICOS. Includes measurements on lung transplant subjects at both week 4 and week 6.

FIG. 11 is a plot showing discrimination between normals (N) and lung transplant Subjects L0=nonrejectors, L1=rejectors) provided by the 2 Genes TNFRSF5 and ICOS. Includes measurements on lung transplant subjects at week 4.

FIG. 12 is a plot showing discrimination between normals (N) and lung transplant Subjects L0=nonrejectors, L1=rejectors) provided by the 2 Genes TNFRSF5 and TNFRSF6. Includes measurements on lung transplant subjects at both week 4 and week 6.

FIG. 13 is a plot showing discrimination between normals (N) and lung transplant Subjects L0=nonrejectors, L1=rejectors) provided by the 2 Genes TNFRSF5 and TNFRSF6. Includes measurements on lung transplant subjects at week 6. 100% of Lung Transplants were correctly classified, 93.8% of Normals were correctly classified in this two gene model.

### DETAILED DESCRIPTION OF SPECIFIC EMBODIMENTS

### Definitions

The following terms shall have the meanings indicated unless the context otherwise requires:

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are tracked to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

“Accuracy” is measure of the strength of the relationship between true values and their predictions. Accordingly, accuracy provided a measurement on how close to a true or accepted value a measurement lies.

“Autoimmune Disorder” includes diseases characterized by abnormal functioning of the immune system that causes your immune system to produce antibodies against your own tissues. Autoimmune disease include for example autoimmune diabetes, growth-onset diabetes, IDDM, insulin-dependent diabetes mellitus, juvenile diabetes, juvenile-onset diabetes, ketoacidosis-prone diabetes, ketosis-prone diabetes, type I diabetes—severe diabetes mellitus with an early onset; catrophic arthritis, rheumatoid arthritis, rheumatism ankylosing spondylitis, Marie-Strumpell disease, rheumatoid spondylitis discoid lupus erythematosus, Hashimoto's disease lupus erythematosus, dermatosclerosis, scleroderma idiopathic thrombocytopenic purpura, purpura hemorrhagica, thrombocytopenic purpura, and Werlhof s disease.

A “baseline profile data set” is a set of values associated with constituents of a Gene Expression Panel resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “biological condition” of a subject is the condition of the subject in a pertinent realm that is under observation, and such realm may include any aspect of the subject capable of being monitored for change in condition, such as health, disease including cancer; autoimmune condition; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Bodily fluid” of a subject includes blood, urine, spinal fluid, lymph, mucosal secretions, prostatic fluid, semen, haemolymph or any other bodily fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

A “composition” includes a chemical compound, a nutriceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel either (i) by direct measurement of such constituents in a biological sample or (ii) by measurement of such constituents in a second biological sample that has been exposed to the original sample or to matter derived from the original sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel resulting from evaluation of a biological sample (or population or set of samples).

A “Gene Expression Profile Inflammatory Index” is the value of an index function that provides a mapping from an instance of a Gene Expression Profile into a single-valued measure of inflammatory condition.

The “health” of a subject includes mental, emotional, physical, spiritual, allopathic, naturopathic and homeopathic condition of the subject.

“Immunosuppression” is the reduction of the activation or efficacy of the immune system. Immunosuppression can self-regulated by the immune system. Immunosuppression can be induced by an infectious agent such as a virus, e.g., HIV. Alternatively, immunosuppression is medically induced by drugs.

“Immunosuppressive drugs” include for example, glucorticoids, cytostatics, antibodies, cyclosporine, tacrolimus, sirolimus, interferons, TNF binding proteins, or mycophenolate.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response, initiated or sustained by any number of chemical, physical or biological agents or combination of agents.

“Inflammatory state” is used to indicate the relative biological condition of a subject resulting from inflammation, or characterizing the degree of inflammation

A “large number” of data sets based on a common panel of genes is a number of data sets sufficiently large to permit a statistically significant conclusion to be drawn with respect to an instance of a data set based on the same panel.

A “normative” condition of a subject to whom a composition is to be administered means the condition of a subject before administration, even if the subject happens to be suffering from a disease.

A “normal” subject is a subject known not to be suffering transplant rejection, an inflammatory condition related to transplant rejection, or immunosuppression, (e.g., normal, healthy individual(s).

A “panel” of genes is a set of genes including at least two constituents.

A “population of cells” refers to any group of cells wherein there is an underlying commonality or relationship between the members in the population of cells, including a group of cells taken from an organism or from a culture of cells or from a biopsy, for example,

A “sample” from a subject may include a single cell or multiple cells or fragments of cells or an aliquot of bodily fluid, taken from the subject, by means including venipuncture, excretion, ejaculation, massage, biopsy, needle aspirate, lavage sample, scraping, surgical incision or intervention or other means known in the art.

A “set” or “population” of samples or subjects refers to a defined or selected group of samples or subjects wherein there is an underlying commonality or relationship between the members included in the set or population of samples or subjects.

A “Signature Profile” is an experimentally verified subset of a Gene Expression Profile selected to discriminate a biological condition, agent or physiological mechanism of action.

A “Signature Panel” is a subset of a Gene Expression Panel, the constituents of which are selected to permit discrimination of a biological condition, agent or physiological mechanism of action.

A “subject” is a cell, tissue, or organism, human or non-human, whether in vivo, ex vivo or in vitro, under observation. As used herein, reference to evaluating the biological condition of a subject based on a sample from the subject, includes using blood or other tissue sample from a human subject to evaluate the human subject's condition; it also includes, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of cancer with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

“Transplant rejection” includes rejection of the donor organ, tissue or cell by the transplant recipient's immune system. “Acute Transplant Rejection” includes a hyper-acute rejection that occurs within minute or hours after graft implantation. “Chronic Transplant Rejection” includes pathologic tissue remodeling resulting in reduced blood flow to tissue, ischemia, fibrosis, and cell death.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels (Precision Profiles™) for the evaluation of (i) biological condition (including with respect to health and disease) and (ii) the effect of one or more agents on biological condition (including with respect to health, toxicity, therapeutic treatment and drug interaction).

In particular, Gene Expression Panels (Precision Profiles™) may be used for measurement of therapeutic efficacy of natural or synthetic compositions or stimuli that may be formulated individually or in combinations or mixtures for a range of targeted biological conditions; prediction of toxicological effects and dose effectiveness of a composition or mixture of compositions for an individual or for a population or set of individuals or for a population of cells; determination of how two or more different agents administered in a single treatment might interact so as to detect any of synergistic, additive, negative, neutral or toxic activity; performing pre-clinical and clinical trials by providing new criteria for pre-selecting subjects according to informative profile data sets for revealing disease status; and conducting preliminary dosage studies for these patients prior to conducting phase 1 or 2 trials. These Gene Expression Panels (Precision Profiles™) may be employed with respect to samples derived from subjects in order to evaluate their biological condition.

The present invention provides Gene Expression Panels (Precision Profiles™) for the evaluation of transplant rejection, inflammatory condition related to transplant rejection and immunosuppression. Immunosuppression is naturally induced, induced by infectious agents, e.g., viruses such as HIV, and medically induced by the administration of drugs that are known to suppress immune function. Medically induced immunosuppression is used in the management of graft rejection post transplant and in the management and treatment of autoimmune disorders. In addition, the Gene Expression Profiles described herein also provided the evaluation of the effect of one or more agents for the treatment of transplant rejection, inflammatory condition related to transplant rejection, and immunosuppressive agents.

The Gene Expression Panels (Precision Profiles™) are referred to herein as the “Precision Profile™ for Transplant Rejection” and the “Precision Profile™ for Immunosuppression”. A Precision Profile™ for Transplant Rejection includes one or more genes, e.g., constituents, listed in Table 1. A Precision Profile™ for Immunosuppression includes one or more genes, e.g., constituents, listed in Table 2. Each gene of the Precision Profile™ for Transplant Rejection and Precision Profile™ for Immunosuppression is referred to herein as a transplant rejection (TX) associated gene or a TX-associated constituent.

The evaluation or characterization of a subject with transplant rejection, an inflammatory condition related to transplant rejection, or immunosuppression, is defined to be diagnosing transplant rejection, an inflammatory condition related to transplant rejection, or immunosuppression, assessing the risk of developing transplant rejection, an inflammatory condition related to transplant rejection, or immunosuppression, or assessing the prognosis of a subject with transplant rejection, an inflammatory condition related to transplant rejection, or immunosuppression. Similarly, the evaluation or characterization of an agent for treatment of transplant rejection, an inflammatory condition related to transplant rejection, or immunosuppressive agents includes identifying agents suitable for the treatment of transplant rejection, an inflammatory condition related to transplant rejection, or suitable for immunosuppression. The agents can be compounds known to treat transplant rejection or an inflammatory condition related to transplant rejection, or compounds that have not been shown to treat transplant rejection or an inflammatory condition related to transplant rejection, compounds known to induce immunosuppression, or compounds that have not been shown to induce immunosuppression.

The agent to be evaluated or characterized for the treatment of transplant rejection or inflammatory conditions related to transplant rejection, or immunosuppressive agents include but are not limited to glucorticoids, cytostatics, antibodies, cyclosporine, tacrolimus, sirolimus, interferons, TNF binding proteins, or mycophenolate.

Transplant rejection, an inflammatory condition related to transplant rejection, or immunosuppression, is evaluated by determining the level of expression (e.g., a quantitative measure) of one or more TX-associated genes. The level of expression is determined by any means known in the art, such as for example quantitative PCR. The measurement is obtained under conditions that are substantially repeatable. Optionally, the qualitative measure of the constituent is compared to a baseline level (e.g. baseline profile set). A baseline level is a level of expression of the constituent in one or more subjects known not to be suffering transplant rejection, an inflammatory condition related to transplant rejection, or immunosuppression, (e.g., normal, healthy individual(s)). Alternatively, the baseline level is derived from one or more subjects known to be suffering from transplant rejection, an inflammatory condition related to transplant rejection. Optionally, the baseline level is derived from the same subject from which the first measure is derived. For example, the baseline is taken from a subject prior to receiving treatment for transplant rejection, an inflammatory condition related to transplant rejection, or at different time periods during a course of treatment. Such methods allow for the evaluation of a particular treatment for a selected individual. Comparison can be performed on test (e.g., patient) and reference samples (e.g., baseline) measured concurrently or at temporally distinct times. An example of the latter is the use of compiled expression information, e.g., a gene expression database, which assembles information about expression levels of TX-associated genes.

A change in the expression pattern in the patient-derived sample of a TX-associated gene compared to the normal baseline level indicates that the subject is suffering from or is at risk of developing transplant rejection or an inflammatory condition related to transplant rejection. In contrast, when the methods are applied prophylacticly, a similar level compared to the normal control level in the patient-derived sample of a TX-associated gene indicates that the subject is not suffering from or is at risk of developing transplant rejection or an inflammatory condition related to transplant rejection. Whereas, a similarity in the expression pattern in the patient-derived sample of a TX-associated gene compared to the baseline level indicates that the subject is suffering from or is at risk of developing transplant rejection or an inflammatory condition related to transplant rejection.

Expression of an effective amount of a TX-associated gene also allows for the course of treatment of transplant rejection, or an inflammatory condition related to transplant rejection to be monitored. In this method, a biological sample is provided from a subject undergoing treatment, e.g., if desired, biological samples are obtained from the subject at various time points before, during, or after treatment for transplant rejection or an inflammatory condition related to transplant rejection. Expression of an effective amount of a TX-associated gene is then determined and compared to baseline profile. The baseline profile may be taken or derived from one or more individuals who have been exposed to the treatment. Alternatively, the baseline level may be taken or derived from one or more individuals who have not been exposed to the treatment. For example, samples may be collected from subjects who have received initial treatment for transplant rejection or an inflammatory condition related to transplant rejection and subsequent treatment for transplant rejection or an inflammatory condition related to transplant rejection to monitor the progress of the treatment.

Differences in the genetic makeup of individuals can result in differences in their relative abilities to metabolize various drugs. Accordingly, the Precision Profile™ for Transplant Rejection (Table 1) and the Precision Profile™ for Immunosuppression (Table 2), disclosed herein, allow for a putative therapeutic or prophylactic to be tested from a selected subject in order to determine if the agent is suitable for treating or preventing transplant rejection or an inflammatory condition related to transplant rejection in the subject. Additionally, other genes known to be associated with toxicity may be used. By suitable for treatment is meant determining whether the agent will be efficacious, not efficacious, or toxic for a particular individual. By toxic it is meant that the manifestations of one or more adverse effects of a drug when administered therapeutically. For example, a drug is toxic when it disrupts one or more normal physiological pathways.

To identify a therapeutic that is appropriate for a specific subject, a test sample from the subject is exposed to a candidate therapeutic agent, and the expression of one or more of TX-associated genes is determined. A subject sample is incubated in the presence of a candidate agent and the pattern of TX-associated gene expression in the test sample is measured and compared to a baseline profile, e.g., a TX baseline profile or a non-TX baseline profile or an index value. The test agent can be any compound or composition. For example, the test agent is a compound known to be useful in the treatment of transplant rejection or an inflammatory condition related to transplant rejection, or as an immunosuppressive agent. Alternatively, the test agent is a compound that has not previously been used to treat transplant rejection or an inflammatory condition related to transplant rejection, or as an immunosuppressive agent.

If the reference sample, e.g., baseline is from a subject that does not have transplant rejection or an inflammatory condition related to transplant rejection, a similarity in the pattern of expression of TX-associated genes in the test sample compared to the reference sample indicates that the treatment is efficacious. Whereas a change in the pattern of expression of TX-associated genes in the test sample compared to the reference sample indicates a less favorable clinical outcome or prognosis.

By “efficacious” is meant that the treatment leads to a decrease of a sign or symptom of transplant rejection or an inflammatory condition related to transplant rejection in the subject or a change in the pattern of expression of a TX-associated gene in such that the gene expression pattern has an increase in similarity to that of a normal baseline pattern. Assessment of transplant rejection or an inflammatory condition related to transplant rejection is made using standard clinical protocols. Efficacy is determined in association with any known method for diagnosing or treating transplant rejection or an inflammatory condition related to transplant rejection.

Agents that are toxic for a specific subject are identified by exposing a test sample from the subject to a candidate agent, and the expression of one or more of TX-associated genes is determined. A subject sample is incubated in the presence of a candidate agent and the pattern of TX-associated gene expression in the test sample is measured and compared to a baseline profile, e.g., a TX-baseline profile or a non-TX baseline profile or an index value. The test agent can be any compound or composition. For example, the test agent is a compound known to be useful in the treatment of transplant rejection or an inflammatory condition related to transplant rejection. Alternatively, the test agent is a compound that has not previously been used to treat transplant rejection or an inflammatory condition related to transplant rejection.

If the reference sample, e.g., baseline is from a subject in whom the candidate agent is not toxic a similarity in the pattern of expression of TX-associated genes in the test sample compared to the reference sample indicates that the candidate agent is not toxic for the particular subject. Whereas a change in the pattern of expression of TX-associated genes in the test sample compared to the reference sample indicates that the candidate agent is toxic.

A Gene Expression Panel (Precision Profile™) is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

It has been discovered that valuable and unexpected results may be achieved when the quantitative measurement of constituents is performed under repeatable conditions (within a degree of repeatability of measurement of better than twenty percent, preferably ten percent or better, more preferably five percent or better, and more preferably three percent or better). For the purposes of this description and the following claims, a degree of repeatability of measurement of better than twenty percent may be used as providing measurement conditions that are “substantially repeatable”. In particular, it is desirable that, each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

Additional embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting Phase 1 or 2 trials.

Gene expression profiling and the use of index characterization for a particular condition or agent or both may be used to reduce the cost of Phase 3 clinical trials and may be used beyond Phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The Subject

The methods disclosed here may be applied to cells of humans, mammals or other organisms without the need for undue experimentation by one of ordinary skill in the art because all cells transcribe RNA and it is known in the art how to extract RNA from all types of cells.

A subject can include those who have not been previously diagnosed as having transplant rejection or an inflammatory condition related to transplant. Alternatively, a subject can also include those who have already been diagnosed as having transplant rejection or an inflammatory condition related to transplant rejection. Optionally, the subject has been previously treated with therapeutic agents, or with other therapies and treatment regimens for transplant rejection or an inflammatory condition related to transplant rejection. For example the subject has been treated with immunosuppressive agents. A subject can also include those who are suffering from, or at risk of developing transplant rejection or an inflammatory condition related to transplant rejection, such as those who exhibit have recently received and organ transplant. A subject can include those who are candidates for immunosuppressive therapy.

### Selecting Constituents of a Gene Expression Panel (Precision Profile™)

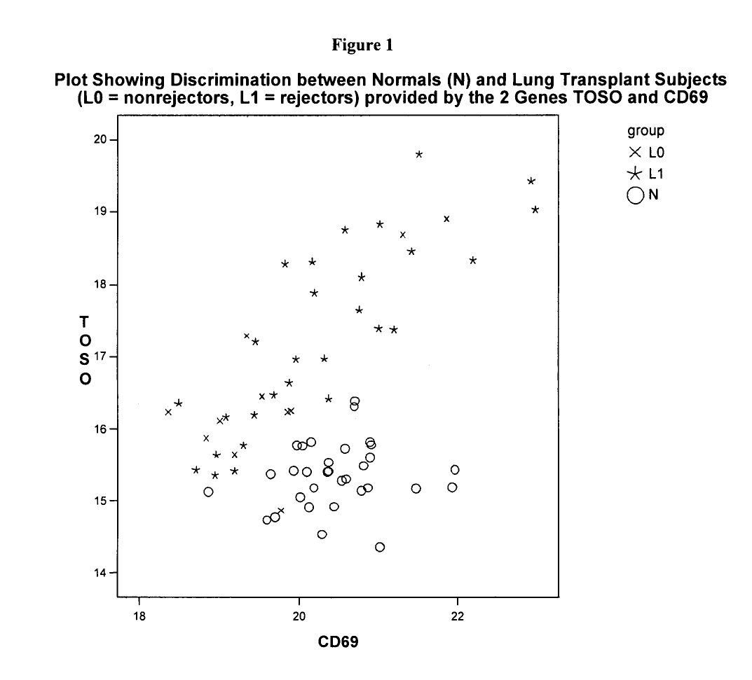

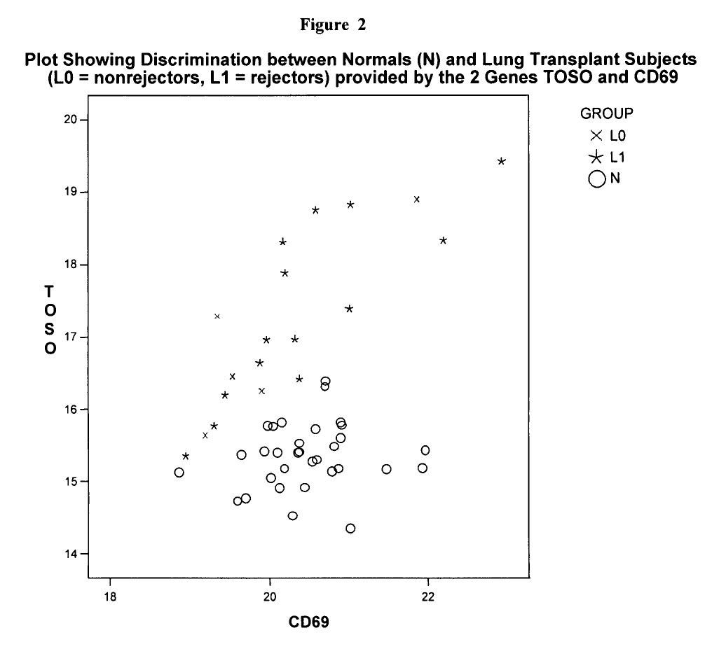

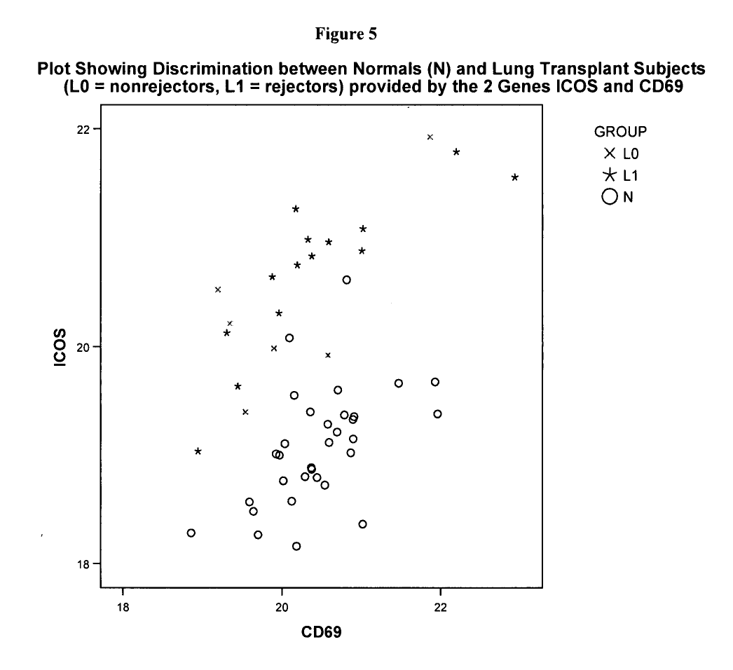

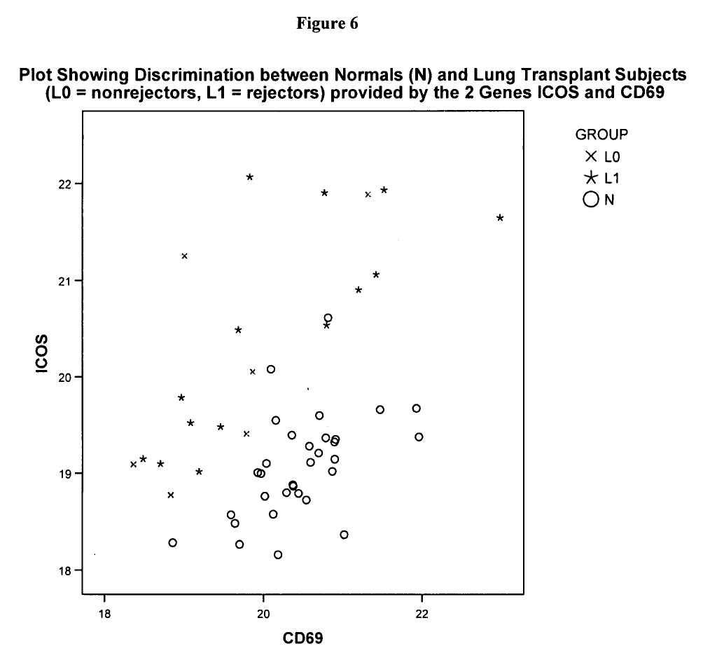

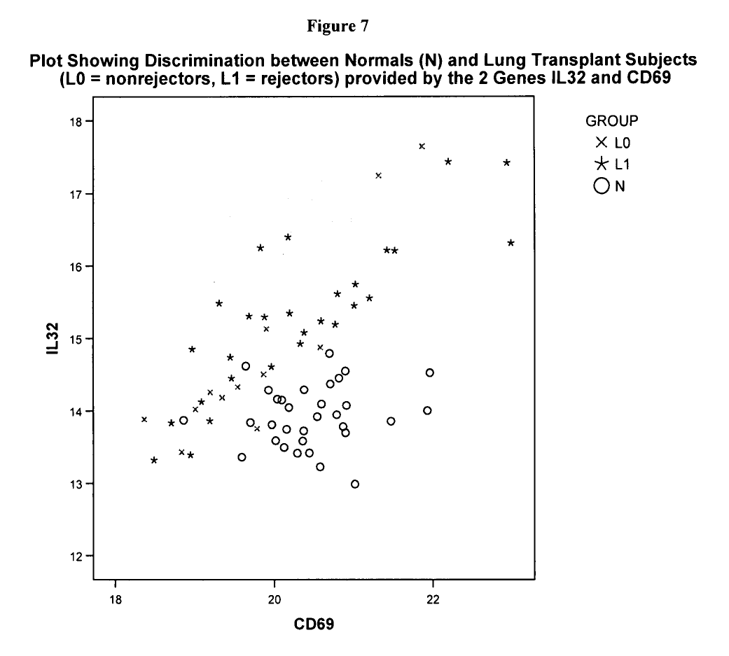

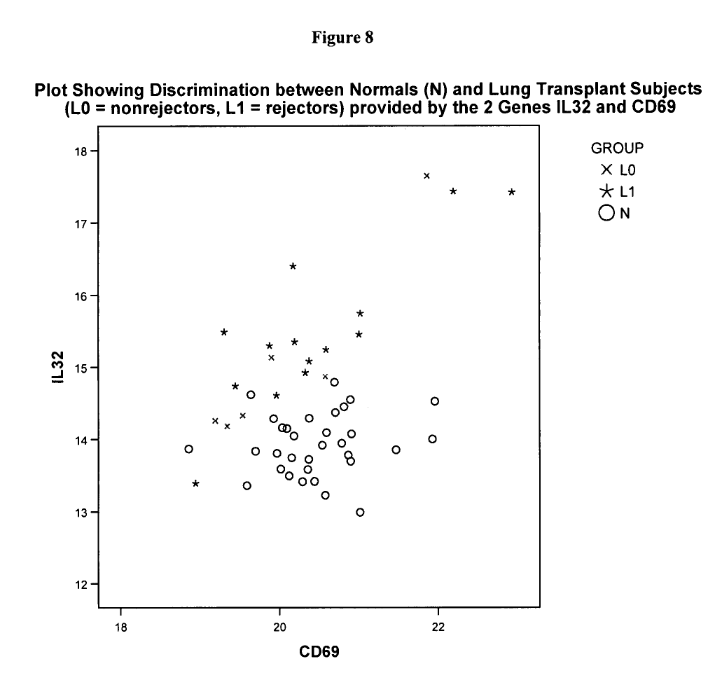

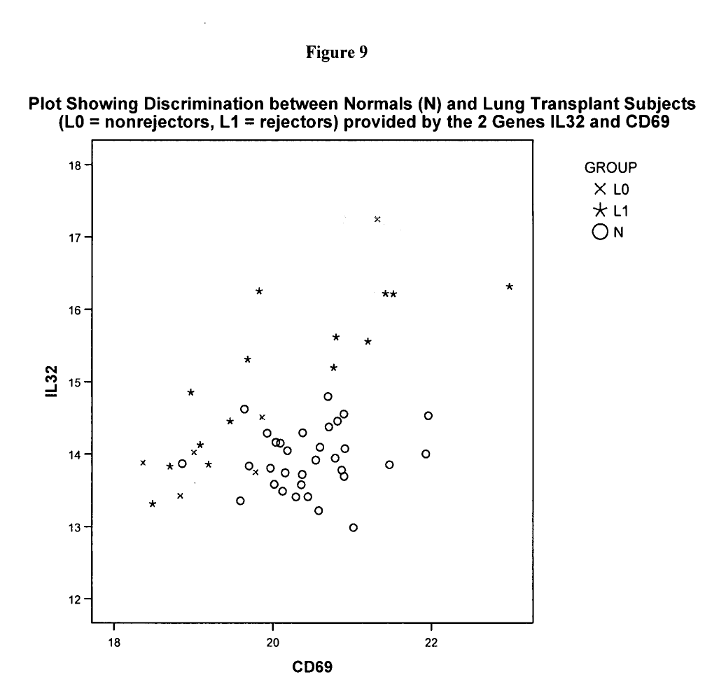

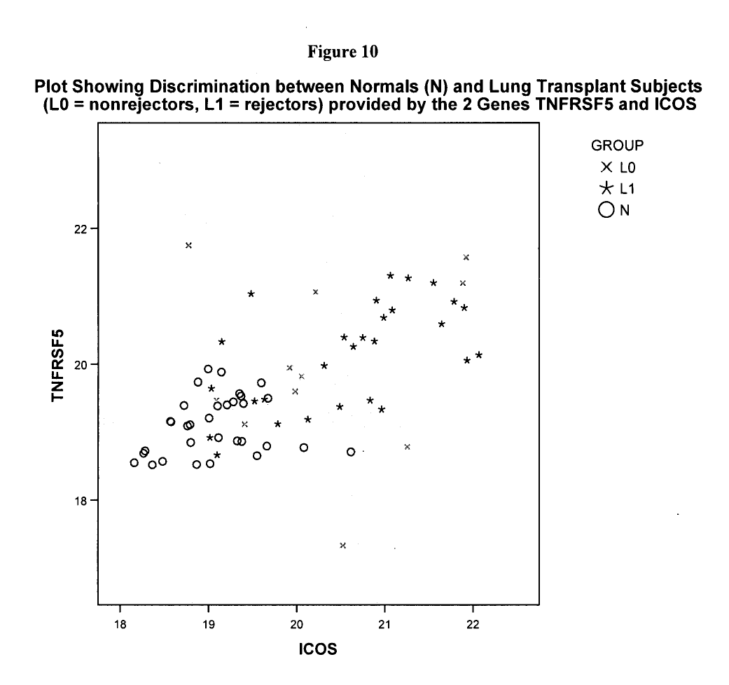

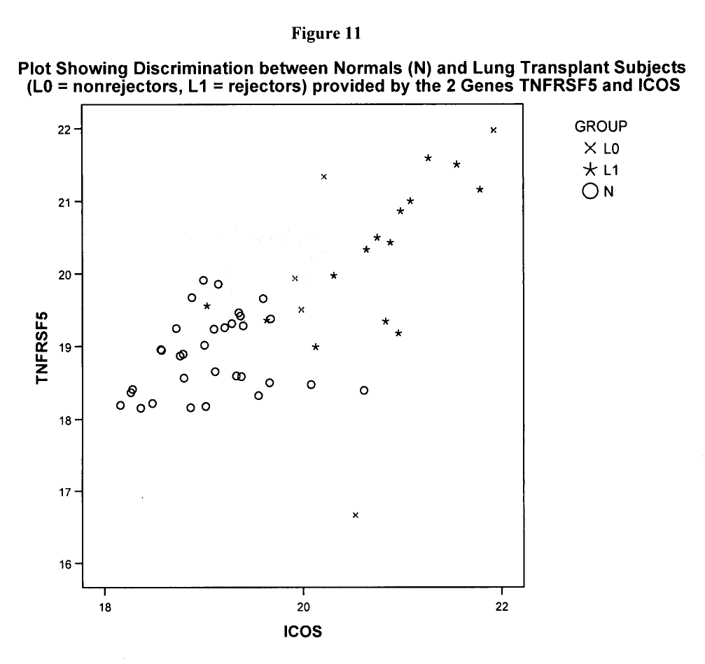

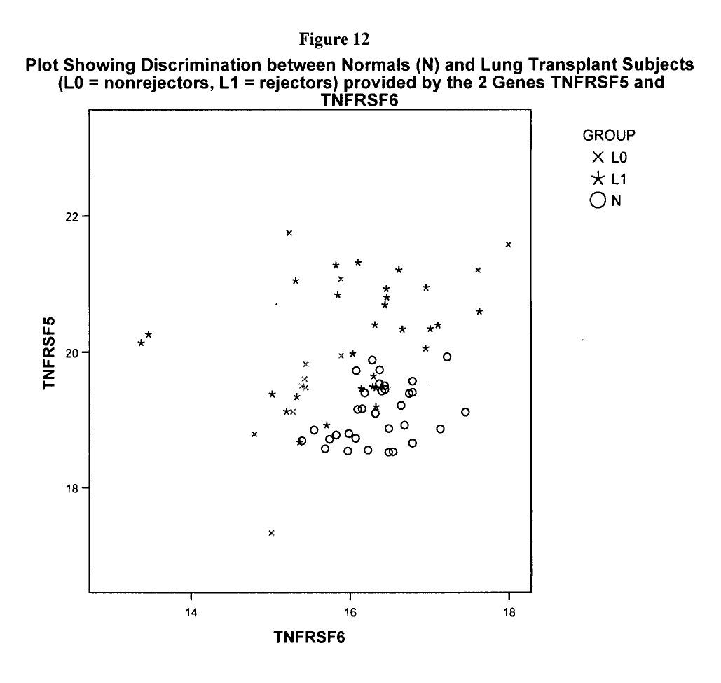

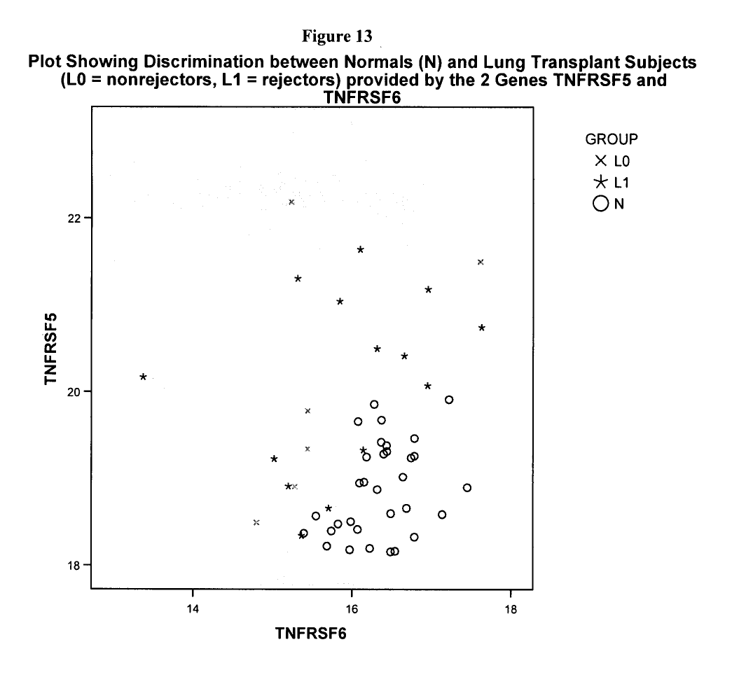

The general approach to selecting constituents of a Gene Expression Panel has been described in PCT application publication number WO 01/25473, incorporated herein in its entirety. A wide range of Gene Expression Panels (Precision Profiles™) have been designed and experimentally validated, each panel providing a quantitative measure of biological condition, that is derived from a sample of blood or other tissue. For each panel, experiments have verified that a Gene Expression Profile using the panel's constituents is informative of a biological condition. (It has also been demonstrated that in being informative of biological condition, the Gene Expression Profile is used, among other things, to measure the effectiveness of therapy, as well as to provide a target for therapeutic intervention.) Tables 1, 2,3,4,5, or 6 listed below, include relevant genes which may be selected for a given Gene Expression Panel (Precision Profiles™), such as the Precision Profiles™ demonstrated herein to be useful in the evaluation of transplant rejection and inflammatory condition related to transplant rejection. Tables 1-6 described below were derived from a study of gene expression patterns described in Examples 1 and 3 below. Table 1 is the Precision Profile™ for Transplant Rejection, a panel of 78 genes whose expression is associated with transplant rejection or inflammatory conditions related to transplant rejection. Table 2 is the Precision Profile™ for Immunosuppression, a panel of 44 genes whose expression is associated with transplant rejection or an inflammatory condition related to transplant rejection. Tables 3-6 and FIGS. 1-13 describe 2 gene models based on genes from the Precision Profile™ for Immunosuppression derived from latent class modeling of the subjects from this study to distinguish from subjects having transplant rejection or an inflammatory condition related to transplant rejection and normal subjects. For example, as shown in FIG. 2, the 2-gene model, TOSO and CD69 correctly classifies lung transplant subjects with 95% accuracy, and normal subjects with 100% accuracy. In general, panels may be constructed and experimentally validated by one of ordinary skill in the art in accordance with the principles articulated in the present application.

### Design of Assays

Typically, a sample is run through a panel in replicates of three for each target gene (assay); that is, a sample is divided into aliquots and for each aliquot the concentrations of each constituent in a Gene Expression Panel (Precision Profile™) is measured. From over a total of 900 constituent assays, with each assay conducted in triplicate, an average coefficient of variation was found (standard deviation/average)*100, of less than 2 percent among the normalized ΔCt measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra-assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue, bodily fluid, cell, or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 90.0 to 100%+/−5% relative efficiency, typically 99.8 to 100% relative efficiency). For example, in determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Whole Blood for Ex Vivo Assessment of a Biological Condition Affected by an Agent.

Human blood is obtained by venipuncture and prepared for assay by separating samples for baseline, no exogenous stimulus, and pro-cancer stimulus with sufficient volume for at least three time points. Typical pro-cancer stimuli include for example, ionizing radiation, free radicals or DNA damaging agents, and may be used individually or in combination. The aliquots of heparinized, whole blood are mixed with additional test therapeutic compounds and held at 37° C. in an atmosphere of 5% CO₂ for 30 minutes. Stimulus is added at varying concentrations, mixed and held loosely capped at 37° C. for the prescribed timecourse. At defined time-points, cells are lysed and RNA extracted by various standard means.

Nucleic acids, RNA and or DNA are purified from cells, tissues or fluids of the test population of cells or indicator cell lines. RNA is preferentially obtained from the nucleic acid mix using a variety of standard procedures (or RNA Isolation Strategies, pp. 55-104, in RNA Methodologies A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit, Catalog #1912, version 9908; Austin, Tex.).

In accordance with one procedure, the whole blood assay for Gene Expression Profiles determination was carried out as follows: Human whole blood was drawn into 10 mL Vacutainer tubes with Sodium Heparin. Blood samples were mixed by gently inverting tubes 4-5 times. The blood was used within 10-15 minutes of draw. In the experiments, blood was diluted 2-fold, i.e. per sample per time point, 0.6 mL whole blood+0.6 mL stimulus. The assay medium was prepared and the stimulus added as appropriate.

A quantity (0.6 mL) of whole blood was then added into each 12×75 mm polypropylene tube. 0.6 mL of 2×LPS (from E. coli serotype 0127:B8, Sigma#L3880 or serotype 055, Sigma #L4005, 10 ng/mL, subject to change in different lots) into LPS tubes was added. Next, 0.6 mL assay medium was added to the “control” tubes. The caps were closed tightly. The tubes were inverted 2-3 times to mix samples. Caps were loosened to first stop and the tubes incubated at 37° C., 5% CO₂ for 6 hours. At 6 hours, samples were gently mixed to resuspend blood cells, and 0.15 mL was removed from each tube (using a micropipettor with barrier tip), and transferred to 0.15 mL of lysis buffer and mixed. Lysed samples were extracted using an ABI 6100 Nucleic Acid Prepstation following the manufacturer's recommended protocol.

The samples were then centrifuged for 5 min at 500×g, ambient temperature (IEC centrifuge or equivalent, in microfuge tube adapters in swinging bucket), and as much serum from each tube was removed as possible and discarded. Cell pellets were placed on ice; and RNA extracted as soon as possible using an Ambion RNAqueous kit.

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies A laboratory guide for isolation and characterization, 2nd edition, 1998, Robert E. Farrell, Jr., Ed., Academic Press; or Chapter 22 pp. 143-151, RNA isolation and characterization protocols, Methods in molecular biology, Volume 86, 1998, R. Rapley and D. L. Manning Eds., Human Press, or 14 in Statistical refinement of primer design parameters, Chapter 5, pp. 55-72, PCR applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular methods for virus detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers. In the present case, amplified cDNA is detected and quantified using the ABI Prism 7900 Sequence Detection System obtained from Applied Biosystems (Foster City, Calif.). Amounts of specific RNAs contained in the test sample or obtained from the indicator cell lines can be related to the relative quantity of fluorescence observed (see for example, Advances in quantitative PCR technology: 5′ nuclease assays, Y. S. Lie and C. J. Petropolus, Current Opinion in Biotechnology, 1998, 9:43-48, or Rapid thermal cycling and PCR kinetics, pp. 211-229, chapter 14 in PCR applications: protocols for functional genomics, M. A. Innis, D. H. Gelfand and J. J. Sninsky, Eds., 1999, Academic Press).

As a particular implementation of the approach described here in detail is a procedure for synthesis of first strand cDNA for use in PCR. This procedure can be used for both whole blood RNA and RNA extracted from cultured cells (i.e. THP-1 cells).

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent).

Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| | |
|---|---|
| 1 reaction (mL) | 11X, e.g. 10 samples (μL) |

| | | | |
|---|---|---|---|
| 10X RT Buffer | 10.0 | 110.0 | |
| 25 mM MgCl₂ | 22.0 | 242.0 | |
| dNTPs | 20.0 | 220.0 | |
| Random Hexamers | 5.0 | 55.0 | |
| RNAse Inhibitor | 2.0 | 22.0 | |
| Reverse Transcriptase | 2.5 | 27.5 | |
| Water | 18.5 | 203.5 | |
| Total: | 80.0 | 880.0 | (80 μL per sample) |

4. Bring each RNA sample to a total volume of 20 μL in a 1.5 mL microcentrifuge tube (for example, for THP-1 RNA, remove 10 μL RNA and dilute to 20 μL with RNase/DNase free water, for whole blood RNA use 20 μL total RNA) and add 80 μL RT reaction mix from step 5,2,3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and β-actin.

The use of the primer probe with the first strand cDNA as described above to permit measurement of constituents of a Gene Expression Panel (Precision Profile™) is as follows:

Materials

1. 20× Primer/Probe Mix for each gene of interest.

2. 20× Primer/Probe Mix for 18S endogenous control.

3. 2× Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism 7700 or 7900 Sequence Detector.

Methods

1. Make stocks of each Primer/Probe mix containing the Primer/Probe for the gene of interest, Primer/Probe for 18S endogenous control, and 2×PCR Master Mix as follows. Make sufficient excess to allow for pipetting error e.g., approximately 10% excess.

The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 95 μL of cDNA into 2000 L of water. The amount of cDNA is adjusted to give Ct values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:
I. To run a QPCR assay in duplicate on the Cepheid SmartCycler® instrument containing three target genes and one reference gene, the following procedure should be followed.

A. With 20× Primer/Probe Stocks.

Materials
1. SmartMix™-HM lyophilized Master Mix.
2. Molecular grade water.
3. 20× Primer/Probe Mix for the 18S endogenous control gene. The endogenous control gene will be dual labeled with VIC-MGB or equivalent.
4. 20× Primer/Probe Mix for each for target gene one, dual labeled with FAM-BHQ1 or equivalent.
5. 20× Primer/Probe Mix for each for target gene two, dual labeled with Texas Red-BHQ2 or equivalent.
6. 20× Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

Methods
1. For each cDNA sample to be investigated, add the following to a sterile 650 PL tube.

| | | |
|---|---|---|
| SmartMix ™-HM lyophilized Master Mix | 1 | bead |
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

Materials
1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| SmartBead ™ containing four primer/probe sets | 1 | bead |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene CT value between 12 and 16.
3. Add 3 μL of the prepared cDNA sample to the reagent mixture bringing the total volume to 50 μL. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.
   II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

Materials
1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

Methods
1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

In other embodiments, any tissue, bodily fluid, or cell(s) (e.g., circulating tumor cells) may be used for ex vivo assessment of a biological condition affected by an agent.

Methods herein may also be applied using proteins where sensitive quantitative techniques, such as an Enzyme Linked ImmunoSorbent Assay (ELISA) or mass spectroscopy, are available and well-known in the art for measuring the amount of a protein constituent. (see WO 98/24935 herein incorporated by reference in its entirety).

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition, e.g., transplant rejection or inflammatory conditions related to transplant rejection. The concept of biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, and/or a particular agent.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects. The baseline profile data set may be normal, healthy baseline.

The profile data set may arise from the same subject for which the first data set is obtained, where the sample is taken at a separate or similar time, a different or similar site or in a different or similar biological condition. For example, a sample may be taken before stimulation or after stimulation with an exogenous compound or substance, such as before or after therapeutic treatment. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library along with or separate from the baseline profile data base and optionally the first profile data set although the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

Selected baseline profile data sets may be also be used as a standard by which to judge manufacturing lots in terms of efficacy, toxicity, etc. Where the effect of a therapeutic agent is being measured, the baseline data set may correspond to Gene Expression Profiles taken before administration of the agent. Where quality control for a newly manufactured product is being determined, the baseline data set may correspond with a gold standard for that product. However, any suitable normalization techniques may be employed. For example, an average baseline profile data set is obtained from authentic material of a naturally grown herbal nutraceutical and compared over time and over different lots in order to demonstrate consistency, or lack of consistency, in lots of compounds prepared for release.

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested. Also found have been repeated instances wherein calibrated profile data sets obtained when samples from a subject are exposed ex vivo to a compound are comparable to calibrated profile data from a sample that has been exposed to a sample in vivo. Importantly, it has been determined that an indicator cell line treated with an agent can in many cases provide calibrated profile data sets comparable to those obtained from in vivo or ex vivo populations of cells. Moreover, it has been determined that administering a sample from a subject onto indicator cells can provide informative calibrated profile data sets with respect to the biological condition of the subject including the health, disease states, therapeutic interventions, aging or exposure to environmental stimuli or toxins of the subject.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within one order of magnitude with respect to similar samples taken from the subject under similar conditions. More particularly, the members may be reproducible within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutraceutical through manufacture, testing and marketing.

The numerical data obtained from quantitative gene expression and numerical data from calibrated gene expression relative to a baseline profile data set may be stored in databases or digital storage mediums and may be retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to transplant rejection or inflammatory conditions related to transplant rejection to be evaluated, with the calibrated profile data set being a comparison between the first profile data set and the baseline profile data set, thereby providing evaluation of transplant rejection or inflammatory conditions related to transplant rejection of the subject.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

For example, a distinct sample derived from a subject being at least one of RNA or protein may be denoted as PI. The first profile data set derived from sample PI is denoted Mj,

where Mj is a quantitative measure of a distinct RNA or protein constituent of PI. The record Ri is a ratio of M and P and may be annotated with additional data on the subject relating to, for example, age, diet, ethnicity, gender, geographic location, medical disorder, mental disorder, medication, physical activity, body mass and environmental exposure. Moreover, data handling may further include accessing data from a second condition database which may contain additional medical data not presently held with the calibrated profile data sets. In this context, data access may be via a computer network.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the transplant rejection or inflammatory conditions related to transplant rejection of the relevant set of subjects by interpreting the calibrated profile data set in the context of at least one other clinical indicator, wherein the at least one other clinical indicator is selected from the group consisting of blood chemistry, X-ray or other radiological or metabolic imaging technique, molecular markers in the blood, other chemical assays, and physical findings.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel (Precision Profile™) that corresponds to the Gene Expression Profile. These constituent amounts form a profile data set, and the index function generates a single value—the index—from the members of the profile data set.

The index function may conveniently be constructed as a linear sum of terms, each term being what is referred to herein as “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ΔCt value for this gene either increases (a positive Ci) or decreases (a lower value) the likelihood of transplant rejection, the ΔCt values of all other genes in the expression being held constant.

The values Cᵢ and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Mass., called Latent Gold®.

Alternatively, other simpler modeling techniques may be employed in a manner known in the art. The index function for transplant rejection may be constructed, for example, in a manner that a greater degree of inflammation (as determined by the profile data set for the Precision Profile™ for Transplant Rejection shown in Table 1 or Precision Profile™ for Immunosuppression shown in Table 2) correlates with a large value of the index function.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is transplant rejection; a reading of 11n this example thus corresponds to a Gene Expression Profile that matches the norm for healthy subjects. A substantially higher reading then may identify a subject experiencing a transplant rejection or an inflammatory condition related to transplant rejection. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that that Gene Expression Profile values (and accordingly constructed indices based on them) tend to be normally distributed, the 0-centered index constructed in this manner is highly informative. It therefore facilitates use of the index in diagnosis of disease and setting objectives for treatment.

Still another embodiment is a method of providing an index that is indicative of transplant rejection or inflammatory conditions related to transplant rejection of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of transplant rejection, the panel including at least two of the constituents of any of the genes listed in the Precision Profile™ for Transplant Rejection (Table 1) or Precision Profile™ for Immunosuppression (Table 2). In deriving the profile data set, such measure for each constituent is achieved under measurement conditions that are substantially repeatable, at least one measure from the profile data set is applied to an index function that provides a mapping from at least one measure of the profile data set into one measure of the presumptive signs of transplant rejection or immunosuppression, so as to produce an index pertinent to transplant rejection; inflammatory conditions related to transplant rejection or immunosuppression of the subject.

As another embodiment of the invention, an index function I of the form

I=C₀+ΣCᵢM₁ᵢ^{P1(i)}M₂ᵢ^{P2(i)},

can be employed, where M₁ and M₂ are values of the member i of the profile data set, Cᵢ is a constant determined without reference to the profile data set, and P1 and P2 are powers to which M₁ and M₂ are raised. The role of P1(i) and P2(i) is to specify the specific functional form of the quadratic expression, whether in fact the equation is linear, quadratic, contains cross-product terms, or is constant. For example, when P1=P2=0, the index function is simply the sum of constants; when P1=1 and P2=0, the index function is a linear expression; when P1=P2=1, the index function is a quadratic expression.

The constant C₀ serves to calibrate this expression to the biological population of interest that is characterized by having transplant rejection or an inflammatory condition related to transplant rejection. In this embodiment, when the index value equals 0, the odds are 50:50 of the subject having transplant rejection vs a normal subject. More generally, the predicted odds of the subject having transplant rejection is [exp(Iᵢ)], and therefore the predicted probability of having transplant rejection is [exp(Iᵢ)]/[1+exp((Iᵢ)]. Thus, when the index exceeds 0, the predicted probability that a subject has transplant rejection is higher than 0.5, and when it falls below 0, the predicted probability is less than 0.5.

The value of C₀ may be adjusted to reflect the prior probability of being in this population based on known exogenous risk factors for the subject. In an embodiment where C₀ is adjusted as a function of the subject's risk factors, where the subject has prior probability pᵢ of having transplant rejection based on such risk factors, the adjustment is made by increasing (decreasing) the unadjusted C₀ value by adding to C₀ the natural logarithm of the ratio of the prior odds of having transplant rejection taking into account the risk factors to the overall prior odds of having transplant rejection without taking into account the risk factors.

### Kits

The invention also includes a TX-detection reagent, i.e., nucleic acids that specifically identify one or more transplant rejection, inflammatory condition related to transplant rejection, or immunosuppression nucleic acids (e.g., any gene listed in Tables 1-6; referred to herein as TX-associated genes or TX-associated constituents) by having homologous nucleic acid sequences, such as oligonucleotide sequences, complementary to a portion of the TX-associated genes nucleic acids or antibodies to proteins encoded by the TX-associated genes nucleic acids packaged together in the form of a kit. The oligonucleotides can be fragments of the TX-associated genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of PCR, a Northern hybridization or a sandwich ELISA as known in the art.

For example, TX-associated genes detection reagents can be immobilized on a solid matrix such as a porous strip to form at least one TX-associated genes detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of TX-associated genes present in the sample. The detection sites may be configured in any suitably detectable shape and are typically in the shape of a bar or dot spanning the width of a test strip.

Alternatively, TX-associated detection genes can be labeled (e.g., with one or more fluorescent dyes) and immobilized on lyophilized beads to form at least one TX-associated gene detection site. The beads may also contain sites for negative and/or positive controls. Upon addition of the test sample, the number of sites displaying a detectable signal provides a quantitative indication of the amount of TX-associated genes present in the sample.

Alternatively, the kit contains a nucleic acid substrate array comprising one or more nucleic acid sequences. The nucleic acids on the array specifically identify one or more nucleic acid sequences represented by TX-associated genes (see Tables 1-6). In various embodiments, the expression of 2, 3, 4, 5, 6, 7, 8, 9, 10, 15, 20, 25, 40 or 50 or more of the sequences represented by TX-associated genes can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, anti sense oligonucleotides, against any of the TX-associated genes in Tables 1-6.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1

### Transplant (TX) Associated Genes

Table 1 lists 78 genes whose expression may be monitored to determine whether a subject will reject an organ transplant. Table 2 lists genes whose expression may be monitored to determine whether an individual is immunosuppressed or the ability of a candidate compound to suppress the immune system.

TABLE 1
Precision Profile ™ for Transplant Rejection

| Gene Symbol | Gene Name | Gene Accession Number |
|---|---|---|
| APAF1 | apoptotic protease activating factor 1 | NM_013229 |
| BAX | BCL2-associated X protein | NM_138761 |
| BCL2 | B-cell CLL/lymphoma 2 | NM_000633 |
| C1QA | Complement component 1, q subcomponent, alpha | NM_015991 |
| | polypeptide | |
| CASP3 | caspase 3, apoptosis-related cysteine peptidase | NM_004346 |
| CCL2 | chemokine (C-C motif) ligand 2 | NM_002982 |
| CCL4 | chemokine (C-C motif) ligand 4 | NM_002984 |
| CCL5 | chemokine (C-C motif) ligand 5 | NM_002985 |
| CCR1 | chemokine (C-C motif) receptor 1 | NM_001295 |
| CCR3 | chemokine (C-C motif) receptor 3 | NM_001837 |
| CD14 | CD14 antigen | NM_000591 |
| CD19 | CD19 Antigen | NM_001770 |
| CD3Z | CD3 Antigen, Zeta Polypeptide | NM_198053 |
| CD4 | CD4 antigen (p55) | NM_000616 |
| CD44 | CD44 antigen (homing function and Indian blood group | NM_000610 |
| | system) | |
| CD86 | CD86 antigen (CD28 antigen ligand 2, B7-2 antigen) | NM_006889 |
| CD8A | CD8 antigen, alpha polypeptide | NM_001768 |
| CSF2 | colony stimulating factor 2 (granulocyte-macrophage) | NM_000758 |
| CSF3 | colony stimulating factor 3 (granulocytes) | NM_000759 |
| CTLA4 | cytotoxic T-lymphocyte-associated protein 4 | NM_005214 |
| CXCL1 | chemokine (C—X—C motif) ligand 1 (melanoma growth | NM_001511 |
| | stimulating activity, alpha) | |
| CXCL10 | chemokine (C—X—C moif) ligand 10 | NM_001565 |
| CXCL2 | Chemokine (C—X—C Motif) Ligand 2 | NM_002089 |
| CXCL9 | chemokine (C—X—C motif) ligand 9 | NM_002416 |
| CXCR3 | chemokine (C—X—C motif) receptor 3 | NM_001504 |
| CXCR4 | chemokine (C—X—C motif) receptor 4 | NM_001008540 |
| CYBB | cytochrome b-245, beta polypeptide (chronic | NM_000397 |
| | granulomatous disease) | |
| EGR1 | early growth response-1 | NM_001964 |
| ELA2 | elastase 2, neutrophil | NM_001972 |
| FCGR1A | Fc fragment of IgG, high affinity receptor IA | NM_000566 |
| HLA-DRB1 | major histocompatibility complex, class II, DR | NM_002124 |
| | beta 1 | |
| HMOX1 | heme oxygenase (decycling) 1 | NM_002133 |
| HSPA1A | heat shock protein 70 | NM_005345 |
| ICAM1 | Intercellular adhesion molecule 1 | NM_000201 |
| ICOS | inducible T-cell co-stimulator | NM_012092 |
| IFI16 | interferon inducible protein 16, gamma | NM_005531 |
| IFNG | interferon gamma | NM_000619 |
| IL10 | interleukin 10 | NM_000572 |
| IL13 | interleukin 13 | NM_002188 |
| IL15 | interleukin 15 | NM_000585 |
| IL18 | interleukin 18 | NM_001562 |
| IL1A | interleukin 1, alpha | NM_000575 |
| IL1B | interleukin 1, beta | NM_000576 |
| IL2 | interleukin 2 | NM_000586 |
| IL4 | interleukin 4 | NM_000589 |
| IL6 | interleukin 6 (interferon, beta 2) | NM_000600 |
| IL7 | interleukin 7 | NM_000880 |
| IL7R | interleukin 7 receptor | NM_002185 |
| IL8 | interleukin 8 | NM_000584 |
| ITGA4 | integrin, alpha 4 (antigen CD49D, alpha 4 subunit of | NM_000885 |
| | VLA-4 receptor) | |
| ITGAM | integrin, alpha M) | NM_000632 |
| MAP3K1 | mitogen-activated protein kinase kinase kinase 1 | XM_042066 |
| MDM2 | Mdm2, transformed 3T3 cell double minute 2, p53 | NM_002392 |
| | binding protein (mouse) | |
| MIF | macrophage migration inhibitory factor (glycosylation- | NM_002415 |
| | inhibiting factor) | |
| MMP9 | matrix metallopeptidase 9 (gelatinase B, 92 kDa | NM_004994 |
| | gelatinase, 92 kDa type IV collagenase) | |
| MPO | myeloperoxidase | NM_000250 |
| MYC | v-myc myelocytomatosis viral oncogene homolog (avian) | NM_002467 |
| NFKB1 | nuclear factor of kappa light polypeptide gene enhancer | NM_003998 |
| | in B-cells 1 (p105) | |
| NFKBIB | nuclear factor of kappa light polypeptide gene enhancer | NM_001001716 |
| | in B-cells inhibitor, beta | |
| NOS2A | nitric oxide synthase 2A (inducible, hepatocytes) | NM_000625 |
| PF4 | platelet factor 4 (Chemokine (C—X—C Motif) Ligand 4) | NM_002619 |
| PI3 | proteinase Inhibitor 3 (Skin Derived) | NM_002638 |
| PRF1 | perforin 1 (pore forming protein) | NM_005041 |
| PRTN3 | proteinase 3 (serine proteinase, neutrophil, Wegener | NM_002777 |
| | granulomatosis autoantigen) | |
| PTPRC | protein tyrosine phosphatase, receptor type, C | NM_002838 |
| PTX3 | pentraxin-related gene, rapidly induced by IL-1 beta | NM_002852 |
| S100A8 | S100 calcium binding protein A8 (calgranulin A) | NM_002964 |
| SERPINE1 | serpin peptidase inhibitor, clade E (nexin, plasminogen | NM_000602 |
| | activator inhibitor type 1), member 1 | |
| SLC7A1 | solute carrier family 7 (cationic amino acid transporter, | NM_003045 |
| | y+ system), member 1 | |
| STAT1 | signal transducer and activator of transcription 1, 91 kDa | NM_007315 |
| STAT3 | signal transducer and activator of transcription 3 (acute- | NM_003150 |
| | phase response factor) | |
| TGFB1 | transforming growth factor, beta 1 (Camurati-Engelmann | NM_000660 |
| | disease) | |
| TNF | tumor necrosis factor (TNF superfamily, member 2) | NM_000594 |
| TNFRSF13B | tumor necrosis factor (ligand) superfamily, member 13b | NM_006573 |
| TNFSF5 | CD40 ligand (TNF superfamily, member 5, hyper-IgM | NM_000074 |
| | syndrome) | |
| TNFSF6 | Fas ligand (TNF superfamily, member 6) | NM_000639 |
| UCP2 | uncoupling protein 2 (mitochondrial, proton carrier) | NM_003355 |
| VEGF | vascular endothelial growth factor | NM_003376 |

TABLE 2
Precision Profile for Immunosuppression

| Gene Symbol | Gene Name | Gene Accession Number |
|---|---|---|
| ADAM17 | a disintegrin and metalloproteinase domain 17 | NM_003183 |
| | (tumor necrosis factor, alpha, converting enzyme) | |
| CCL1 | chemokine (C-C motif) ligand 1 | NM_002981 |
| CCL3 | chemokine (C-C motif) ligand 3 | NM_002983 |
| CCR2 | chemokine (C-C motif) receptor 2 | NM_000647 |
| CCR5 | chemokine (C-C motif) receptor 5 | NM_000579 |
| CD69 | CD69 antigen (p60, early T-cell activation antigen) | NM_001781 |
| CD80 | CD80 antigen (CD28 antigen ligand 1, B7-1 | NM_005191 |
| | antigen) | |
| CDKN1A | cyclin-dependent kinase inhibitor 1A (p21, Cip1) | NM_000389 |
| CYP3A4 | cytochrome P450, family 3, subfamily A, | NM_017460 |
| | polypeptide 4 | |
| DUSP6 | dual specificity phosphatase 6 | NM_001946 |
| GZMB | granzyme B (granzyme 2, cytotoxic T-lymphocyte- | NM_004131 |
| | associated serine esterase 1) | |
| HLA-DRA | major histocompatibility complex, class II, DR alpha | NM_019111 |
| ICOS | inducible T-cell co-stimulator | NM_012092 |
| IFI16 | interferon inducible protein 16, gamma | NM_005531 |
| IL12B | interleukin 12 p40 | NM_002187 |
| IL1R1 | interleukin 1 receptor, type I | NM_000877 |
| IL1RN | interleukin 1 receptor antagonist | NM_173843 |
| IL23A | interleukin 23, alpha subunit p19 | NM_016584 |
| IL2RA | interleukin 2 receptor, alpha | NM_000417 |
| IL32 | interleukin 32 | NM_001012631 |
| IL5 | interleukin 5 (colony-stimulating factor, eosinophil) | NM_000879 |
| IRF1 | interferon regulatory factor 1 | NM_002198 |
| IRF5 | interferon regulatory factor 5 | NM_002200 |
| JAK1 | janus kinase 1 (a protein tyrosine kinase) | NM_002227 |
| JUN | v-jun sarcoma virus 17 oncogene homolog (avian) | NM_002228 |
| LTA | lymphotoxin alpha (TNF superfamily, member 1) | NM_000595 |
| MHC2TA | class II, major histocompatibility complex, | NM_000246 |
| | transactivator | |
| MNDA | myeloid cell nuclear differentiation antigen | NM_002432 |
| PLA2G7 | phospholipase A2, group VII (platelet-activating | NM_005084 |
| | factor acetylhydrolase, plasma) | |
| PLAU | plasminogen activator, urokinase | NM_002658 |
| PLAUR | plasminogen activator, urokinase receptor | NM_002659 |
| PRF1 | perforin 1 (pore forming protein) | NM_005041 |
| PTGS2 | prostaglandin-endoperoxide synthase 2 | NM_000963 |
| | (prostaglandin G/H synthase and cyclooxygenase) | |
| RAF1 | v-raf-1 murine leukemia viral oncogene homolog 1 | NM_002880 |
| SERPINA1 | serine (or cysteine) proteinase inhibitor, clade A | NM_000295 |
| | (alpha-1 antiproteinase, antitrypsin), member 1 | |
| SSI-3 | suppressor of cytokine signaling 3 | NM_003955 |
| STAT1 | signal transducer and activator of transcription 1, | NM_007315 |
| | 91 kDa | |
| THBS1 | thrombospondin 1 | NM_003246 |
| TIMP1 | tissue inhibitor of metalloproteinase 1 | NM_003254 |
| TNFRSF5 | CD40 antigen (TNF receptor superfamily member | NM_152854 |
| | 5) | |
| TNFRSF6 | Fas (TNF receptor superfamily, member 6) | NM_000043 |
| TNFSF10 | tumor necrosis factor (ligand) superfamily, member | NM_003810 |
| | 10 | |
| TOSO | Fas apoptotic inhibitory molecule 3 | NM_005449 |
| TSC22D3 | TSC22 domain family, member 3 | NM_198057 |

### Example 2

### Determination of Genes Differentially Expressed in Acute Lung Transplant Rejection

The objective of this study was to ascertain determine gene expression profiles in acute rejection in lung transplant recipients. To do this, several questions need to be answered. These include: 1) Are there characteristic changes in whole blood gene expression that are pathomnemonic for acute rejection that can be detected in patients who are being treated with significant immunosuppressive therapy? 2) What is the time lag between changes in gene expression and the clinical manifestations of rejection? 3) Can a simple, cost-effective test be developed that can identify these changes in time for an intervention to be initiated without having to corroborate the results with invasive diagnostic procedures? The specific aims of the proposed research were to:
1. Measure the expression of 88 inflammation-immune related genes in whole blood from patients who are about to initiate high-dose immunosuppressive therapy for the treatment of an episode of acute LTx rejection.
2. Compare these data to reference databases of normals and to the patients, themselves, prior to the onset of rejection.
3. Select a subset of these 88 genes coupled with candidate biomedical algorithms for use in future studies designed to test the ability to predict and monitor acute LTx. Measure the expression of 88 inflammation-immune related genes in whole blood from patients who are about to initiate high-dose immunosuppressive therapy for the treatment of an episode of acute LTx rejection.
4. Compare these data to reference databases of normals and to the patients, themselves, prior to the onset of rejection.
5. Select a subset of these 88 genes coupled with candidate biomedical algorithms for use in future studies designed to test the ability to predict and monitor acute LTx

The method is composed of controlled sample collection, high-precision molecular analyses, specific databases, biomedical algorithms, and standard operating practices which deliver mRNA analyses with wide dynamic range for a panel of selected genes. The majority of sample processing is performed robotically to limit deleterious effects of nucleases, especially ribonucleases. The following procedures are well-established in our laboratory, and yield high quality cDNA with highly precise quantitative PCR.

Samples were collected into PAXgene® tubes (PreAnalytiX) to stabilize mRNA levels. These tubes contain agents that inhibit RNase and stop gene transcription at the time of collection. It gas been shown that they are effective for days to weeks at room temperature, and permit storage of blood samples for months or longer when frozen (Rainen, et al., 2002). Samples are frozen immediately following collection to permit batch preparation. RNA is extracted from these samples using the PAXgene accompanying extraction chemistry and procedures. First strand cDNA will be synthesized by reverse transcription following priming with random hexamers, using Applied Biosystems chemistry and an AB Prism 6600 robot. These samples are stored at −70° prior to quantitative PCR.

Quantitative PCR was performed with the aid of AB Prisms 7900 Sequence Detector robots. Primer and probe sets have been engineered by Source to deliver high PCR efficiency and precision. As noted in the Preliminary Results section, these primer/probe sets generate consistently reproducible results with % CVs better than 2% for control sets of cDNA. Using these reagents and procedures, it has been demonstrated that human gene expression in whole blood is highly stable over time and that individuals are remarkably similar to each other, revealing a common pattern of gene expression.

PCR reactions were run in 384-well plates and the intensity of released fluors measured. The end-point of the reaction occurs when the fluorescent intensity just exceeds the sample background (threshold crossing, C_{T}). Samples are multiplexed, so the C_{T} for an constitutively expressed gene will be used to calibrate the reaction. The difference between these values ΔC_{T} are used for further consideration.

To compare samples, the ΔC_{T} for each gene product will be compared to the ΔC_{T} for the corresponding gene product under control conditions (preferably the pre-test expression level for the same individual, but the “normal” pattern value may also be used). This AACT value is exponentially related to the level of gene expression:

relative mRNA=2^{-ΔΔCT}

The genes examined in this study are listed in Tables 1 and 2, above.

To determine whether high-precision molecular analysis of gene expression in whole blood, using 88 gene loci, accurately predicts the occurrence of acute lung transplant rejection gene expression changes in 20 patients who have undergone lung transplantation, following their progress throughout the first 12 weeks post surgery are measured. Therapeutic agents and interventions are subject to the discretion of the attending physician.

Lung transplant patients are routinely examined according to the following schedule:
Enrollment (2 weeks post transplant) (1 sample)
Twice a week for the first month post transplant (4 samples)
Once a week for the following 8 weeks (8 samples)

At these visits, patients undergo tests for complete blood count, comprehensive chemistry panel, cyclosporine level, PA and lateral chest x-rays, spirometry and transbronchial biopsies.

Determination of acute rejection will be defined histologically according to guidelines set by the Lung Rejection Study Group. Acute rejection is classified as follows:

| | |
|---|---|
| Grade 0 (no rejection) | Normal pulmonary parenchyma |
| Grade A1 (minimal) | Rare perivascular mononuclear infiltrates, not |
| | obvious at low power (40X) |
| Grade A2 (mild) | Frequent perivascular mononuclear infiltrates, |
| | easily seen at low power (40X) |
| Grade A3 (moderate) | Dense perivascular cuffing by mononuclear |
| | cells, extension of inflammation into the |
| | interstitium |
| Grade A4 (severe) | Diffuse perivascular, interstitial and air space |
| | infiltrates |

In order to decrease sampling error, 10 transbronchial biopsies will be taken from three different lung segments. A positive endpoint for rejection will be considered as the following:

| | |
|---|---|
| In surveillance biopsies | Definitive histologic evidence of |
| (performed on study | rejection > GradeA2 on |
| days 14, 42, 84, and 180) | transbronchial biopsy |
| In symptomatic patients | Definitive histologic evidence |
| | of any Grade of rejection on transbronchial |
| | biopsy or open lung biopsy |
| | A steroid responsive clinical syndrome |
| | characterized by fever, resting or exercise |
| | oxygen desaturation, a fall in FEV₁ of greater |
| | than 15% or pulmonary infiltrates after |
| | infection had been excluded by |
| | bronchoalveolar lavage (BAL) |

Additional Tests Required for this Study:

The only tests that will be added for participants in this study are the drawing of a 2.5 mL blood sample per visit from each patient. Source Precision Medicine will not be responsible for any of the costs associated with the standard care of the patients; any costs applied to the grant will be for blood sample and data acquisition.

The blood samples are collected into PAXgene™ tubes for gene expression analysis. These samples will be stored according to Source Precision Medicine standardized procedures detailed in Source Precision Medicine internal protocol SC055 until analysis is completed at a later date.

High-precision gene expression analysis is conducted by standard Source Precision Medicine protocols, described briefly above. The study requires analysis of 88 mRNA species for 4 samples taken from each of the patients who undergoes acute rejection during the course of the study. Panels of 88 genes, run in quadruplicate along with internal standards, will require one 384-well plate per sample. Data arising from each sample will be transformed to relative mRNA levels, calibrated to Source normals, and the results stored in a lung transplant-specific database, together with disease-related information collected from the traditional monitoring procedures for lung transplant patients. These data are examined in depth to ascertain whether or not gene expression data is effective for developing predictive biomedical algorithms that can predict the onset of acute rejection.

Based upon the experience of Dr. Martin Zamora's group at the University of Colorado Health Sciences Campus (UCHSC), approximately 65% of lung transplant patients will experience an episode of acute rejection within the first 12 weeks post-surgery. Accordingly, it is predicted that 10-14 of the 20 patients involved in this study will experience such an episode during the course of the study. High-precision molecular analysis on four blood samples per patient suffering acute transplant rejection will be conducted. The samples tested will include:

Sample taken at time of diagnosis of acute rejection,

Sample taken immediately before the diagnosis of rejection,

The next most proximal sample,

The sample most temporally removed from the rejection episode

Traditional and advanced statistical modeling, stepwise regression analysis, and cluster analysis to the both the normal and disease-specific gene expression data has been previously applied. In addition, covariant analysis in which each gene is examined separately and compared to the others, searching for groups of genes with similar patterns of behavior have also been applied. Using latent class modeling, genes are clustered into groups with common characteristics and look for predictive factors. Similar techniques will be used with the LTx data, searching for both absolute and relative signals of rejection.

While a large panel of gene expression products will yield interesting results in the arena of research, analysis of this many genes is likely not required to reliably predict acute lung transplant rejection. Reduction in the number of gene loci to be tested will introduce a corresponding welcome reduction in direct or indirect patient cost.

To reduce the count of gene loci, we will rely on data obtained as the result of completion of Specific Aims #1 and #2. Candidate genes will be selected from the larger panel based on patterns in relation to clinical findings of acute rejection, as detailed above. Each gene locus will be evaluated in test biomedical algorithms to develop indices that accurately predict the onset of rejection. In this study, 88 genes for up to 20 patients and 4 time points will be evaluated. Preliminary algorithms will be developed for the first 6 patients, subsequently tested over the remaining 14. Successive iterations will be required to reach a consensus set of algorithms that can be tested during Phase II research with a larger patient base. Completion of candidate gene loci selection at the end of Phase I will lay the foundation for database population, to be proposed in Phase II of this study.

Human subject involvement in this project is limited to blood donation. The research plan will require up to 13 blood donations from each subject over the course of their first 12 weeks following lung transplantation surgery. Participants will be included in the study up until the time when or if they are diagnosed with acute transplant rejection. Subjects of all races, genders and ages will be enrolled on an availability basis.

Blood was drawn according to standard conditions at Source Precision Medicine. Approximately 35 ml of blood will be collected from each subject over the course of the study. These samples were collected under sterile conditions by medical personnel associated with the University of Colorado, from the antecubital vein via venipuncture into standard blood collection tubes (PAXgene and heparinized). These samples were exclusively for the experiments described. Blood collection and processing are described in the Experimental Design section. Information gathered regarding the patients will be collected on coded forms to ensure anonymity.

### Example 3

### Clinical Data analyzed with Latent Class Modeling

Using Source MDx ΔCt measurements on 44 genes that are known to be involved in suppression of the immune system, strong significant differences were detected between 20 lung transplant (LT) subjects and 32 Normals (i.e., individuals not receiving and organ transplant). Since the LT subjects were given a drug to suppress their immune system, this type of difference is not unexpected, but is much less likely to be detected using less precise measurements.

A stepwise logistic regression was used to evaluate all genes for their ability to discriminate between these 2 groups, separately, as well as in conjunction with other genes. In step 1, the procedure selects the gene that is most significant (lowest p-value) to be the initial gene in the model. In the second step of the procedure, the remaining 43 genes are evaluated to determine their incremental p-values given that the first gene is included in the model. The one that shows the most improvement in the ability of the resulting 2-gene model to discriminate between the 2 groups (lowest incremental p-value) is then added as the 2^{nd} gene in the model. Although this procedure could continue to include more than 2 genes in the model, for these data almost perfect discrimination was found with just 2 genes.

Table 3A shows the results of the first 2 steps. In step 1, TOSO is found to be most significant (p=4.8×10⁻¹²). In the second step CD69 enters into the model. FIG. 1 shows how these 2 genes work together to discriminate between the 2 groups. It is shown that normals have TOSO values less than 16.5, while only a small number of LT subjects do. However, those LT subjects who do, also have much lower values on CD69 than the normals, and hence based on the 2-genes a discrimination line can be added to the plot showing almost perfect separation between the 2 groups. Normals fall below and to the right of the line, LT subjects above and to the right.

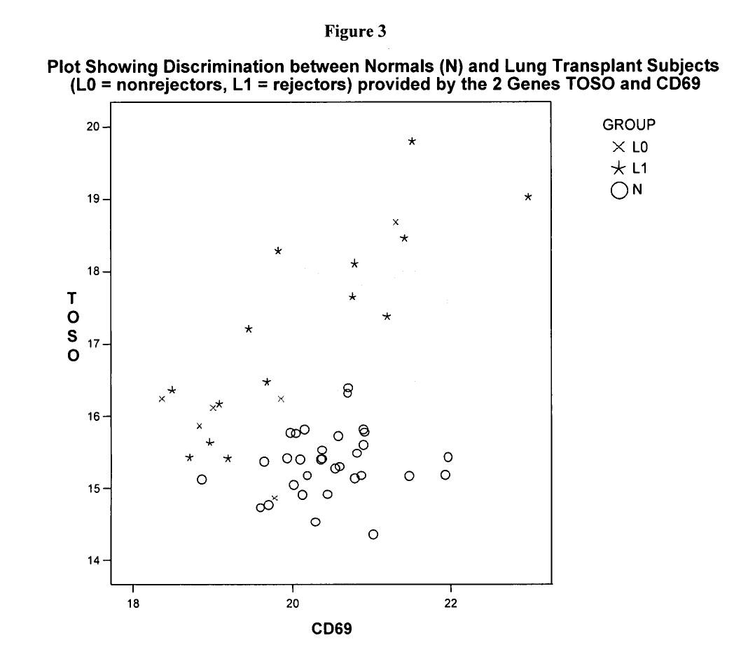

Each LT subject contributes 2 points to this analysis, corresponding to whether the measurement was obtained during week 4 or week 6 following the transplant. Table 3B shows how the results compare if analyses were conducted on week 4 and week 6 LT data separately, where each case contributes only a single point. As shown, the results are very similar, and the same 2 genes are obtained as before regardless of whether week 4 or week 6 measurements are used. Also, in both of these cases the p-values are similar to those shown in Table 3A. FIGS. 2 and 3 show the resulting plots.

Among the LTs, separate symbols are used to distinguish between those who showed a rejection event and those who did not during the 12 weeks following the transplant. As can be seen in FIGS. 1-3, while these 2 genes discriminate between normals and LTs, they do not appear to discriminate between the rejecters and non-rejecters. To see if any of these 48 genes are involved in rejection, the stepwise logistic regression was performed on the LTs, trying to discriminate between the 6 non-rejecters (L0) and the 14 rejecters (L1). No significant differences were found among any of these genes based on week 4 data or week 6 data. This may be due to the small sample sizes of the 2 groups—with such small sample sizes, the statistical power to detect small differences is weak. Or, it may be that these genes are not related to rejection.

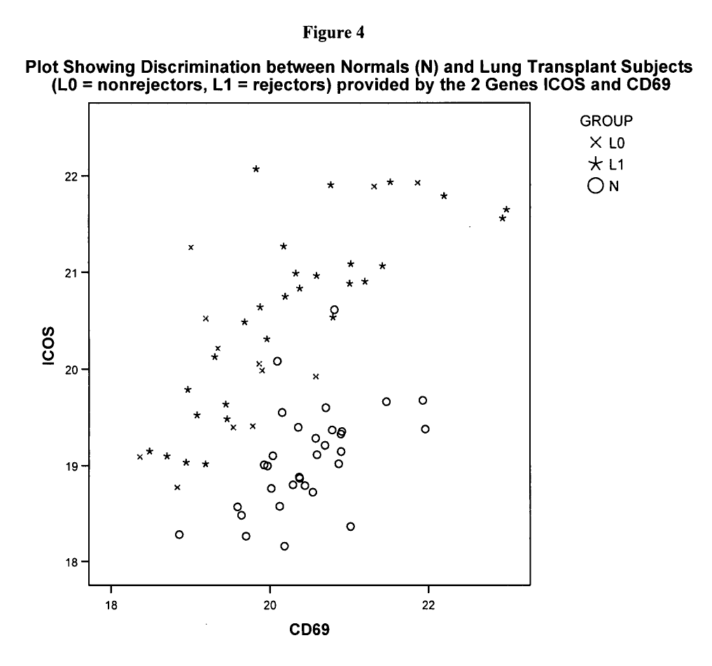

TOSO and CD69 are not the only pair of genes that provide strong discrimination. As shown in the first step of the stepwise procedure in Table 3A (columns labeled “1 gene-model”), the p-values are quite low for many genes. Table 4 shows the resulting 2-gene model when the second most significant gene, ICOS, is used instead of TOSO as the first gene to be included in the model. Again, CD69 turns out to be the second gene in the model. This result occurs whether the analysis is performed using week 4 (left-most portion of Table 4) or week 6 measurements (right-most portion of Table 4). FIGS. 4, 5 and 6 provide plots for this model, corresponding to FIGS. 1, 2 and 3 for the first model, respectively. As a rough measure of goodness of prediction, the R² is shown in the Tables. For comparability across models, these are based on the combined week 4 and 6 data. It is shown that the R² for this 2-gene model is 0.82, which is slightly lower than the 0.84 obtained from the first model.

Several additional alternative 2-gene models are also shown (see Tables 5 and 6). In Table 5, the gene IL32 replaces TOSO (and ICOS) as the first gene, and again CD69 is obtained as the second gene in the model. The corresponding Figures are 7, 8, and 9 for this model. Table 6 shows a model where LTA is the first gene. The second gene turns out to be different depending on whether week 4 or week 6 measurements are used. Hence, we obtain 2 additional alternative 2-gene models here. With weaker 2-gene models, the resulting 2^{nd} gene does not necessarily turn out to be the same.

TABLE 3A
R-squared = 0.84

| 1-gene | | | | | |
|---|---|---|---|---|---|
| model | | | | | |
| weeks 4 & 6 | | p-value | 2-gene model | | |
| TOSO | 1 | 4.80E−12 | TOSO | 1 | 4.80E−12 |
| ICOS | 1 | 1.80E−10 | CD69 | 2 | 3.80E−08 |
| IL23A | 1 | 2.00E−08 | TNFRSF6 | 2 | 5.40E−06 |
| IL32 | 1 | 3.00E−08 | JUN | 2 | 6.40E−06 |
| PLA2G7 | 1 | 1.60E−07 | ADAM17 | 2 | 0.00014 |
| TNFRSF5 | 1 | 3.00E−07 | IL1R1 | 2 | 0.00079 |
| LTA | 1 | 3.50E−07 | CDKN1A | 2 | 0.0016 |
| MHC2TA | 1 | 3.80E−07 | SSI3 | 2 | 0.0051 |
| PRF1 | 1 | 3.50E−06 | DUSP6 | 2 | 0.0056 |
| HLADRA | 1 | 7.60E−06 | PLAU | 2 | 0.0069 |
| CCR5 | 1 | 5.70E−05 | TNFSF10 | 2 | 0.0085 |
| GZMB | 1 | 5.80E−05 | RAF1 | 2 | 0.011 |
| CCL3 | 1 | 6.60E−05 | PLA2G7 | 2 | 0.018 |
| IL1R1 | 1 | 0.00012 | PLAUR | 2 | 0.044 |
| JAK1 | 1 | 0.00016 | TSC22D3 | 2 | 0.06 |
| TSC22D3 | 1 | 0.00021 | SERPINA1 | 2 | 0.062 |
| IL2RA | 1 | 0.0003 | ICOS | 2 | 0.069 |
| PLAU | 1 | 0.0034 | TIMP1 | 2 | 0.088 |
| CCR2 | 1 | 0.0036 | IL2RA | 2 | 0.092 |
| CDKN1A | 1 | 0.0061 | IL12B | 2 | 0.13 |
| CD80 | 1 | 0.018 | IL1RN | 2 | 0.15 |
| SSI3 | 1 | 0.02 | MNDA | 2 | 0.23 |
| IRF5 | 1 | 0.024 | CCL1 | 2 | 0.31 |
| IL5 | 1 | 0.025 | PTGS2 | 2 | 0.34 |
| TNFRSF6 | 1 | 0.027 | CYP3A4 | 2 | 0.37 |
| IL12B | 1 | 0.039 | IRF1 | 2 | 0.37 |
| STAT1 | 1 | 0.075 | CD80 | 2 | 0.42 |
| CD69 | 1 | 0.17 | CCR5 | 2 | 0.44 |
| ADAM17 | 1 | 0.19 | IRF5 | 2 | 0.46 |
| IRF1 | 1 | 0.25 | CCR2 | 2 | 0.5 |
| IFI16 | 1 | 0.32 | GZMB | 2 | 0.5 |
| THBS1 | 1 | 0.38 | PRF1 | 2 | 0.51 |
| TNFSF10 | 1 | 0.43 | THBS1 | 2 | 0.51 |
| SERPINA1 | 1 | 0.44 | IL23A | 2 | 0.53 |
| TIMP1 | 1 | 0.5 | IL32 | 2 | 0.63 |
| CCL1 | 1 | 0.52 | HLADRA | 2 | 0.66 |
| PTGS2 | 1 | 0.54 | STAT1 | 2 | 0.69 |
| IL1RN | 1 | 0.63 | IFI16 | 2 | 0.77 |
| CYP3A4 | 1 | 0.69 | JAK1 | 2 | 0.79 |
| MNDA | 1 | 0.81 | CCL3 | 2 | 0.8 |
| DUSP6 | 1 | 0.86 | MHC2TA | 2 | 0.86 |
| RAF1 | 1 | 0.98 | LTA | 2 | 0.93 |
| PLAUR | 1 | 0.99 | TNFRSF5 | 2 | 0.96 |
| JUN | 1 | 0.99 | IL5 | 2 | 0.98 |

TABLE 3B

| 2-gene | | | | | |
|---|---|---|---|---|---|
| model | | | 2-gene model | | |
| week 4 | | p-value | week 6 | | p-value |
| TOSO | 1 | 1.70E−10 | TOSO | 1 | 4.40E−08 |
| CD69 | 2 | 2.80E−06 | CD69 | 2 | 2.30E−06 |
| IL1R1 | 2 | 2.30E−03 | TNFRSF6 | 2 | 3.00E−06 |
| JUN | 2 | 1.10E−02 | JUN | 2 | 6.40E−06 |
| TNFRSF6 | 2 | 1.50E−02 | ADAM17 | 2 | 0.00012 |
| ADAM17 | 2 | 1.60E−02 | CDKN1A | 2 | 0.0021 |
| ICOS | 2 | 1.80E−02 | PLAU | 2 | 0.0031 |
| TSC22D3 | 2 | 2.70E−02 | SSI3 | 2 | 0.0061 |
| PLA2G7 | 2 | 3.20E−02 | IL1R1 | 2 | 0.0074 |
| TNFSF10 | 2 | 4.20E−02 | DUSP6 | 2 | 0.01 |
| CDKN1A | 2 | 4.80E−02 | RAF1 | 2 | 0.02 |
| SSI3 | 2 | 7.20E−02 | TNFSF10 | 2 | 0.022 |
| DUSP6 | 2 | 7.90E−02 | PLA2G7 | 2 | 0.044 |
| IL12B | 2 | 1.00E−01 | PLAUR | 2 | 0.047 |
| PTGS2 | 2 | 1.10E−01 | PTGS2 | 2 | 0.056 |
| IL32 | 2 | 0.14 | IL2RA | 2 | 0.057 |
| RAF1 | 2 | 0.16 | TIMP1 | 2 | 0.075 |
| LTA | 2 | 0.19 | SERPINA1 | 2 | 0.093 |
| PLAU | 2 | 0.22 | CCL1 | 2 | 0.16 |
| IL23A | 2 | 0.23 | IRF1 | 2 | 0.16 |
| PLAUR | 2 | 0.24 | TSC22D3 | 2 | 0.21 |
| SERPINA1 | 2 | 0.24 | IL1RN | 2 | 0.22 |
| TNFRSF5 | 2 | 0.26 | MNDA | 2 | 0.24 |
| CYP3A4 | 2 | 0.32 | ICOS | 2 | 0.28 |
| IRF5 | 2 | 0.34 | IL12B | 2 | 0.31 |
| IL1RN | 2 | 0.37 | CCR2 | 2 | 0.32 |
| TIMP1 | 2 | 0.4 | LTA | 2 | 0.41 |
| CCR5 | 2 | 0.47 | CD80 | 2 | 0.41 |
| PRF1 | 2 | 0.48 | TNFRSF5 | 2 | 0.51 |
| THBS1 | 2 | 0.52 | CCR5 | 2 | 0.54 |
| IL2RA | 2 | 0.62 | GZMB | 2 | 0.59 |
| CCL1 | 2 | 0.64 | IRF5 | 2 | 0.62 |
| GZMB | 2 | 0.68 | THBS1 | 2 | 0.67 |
| JAK1 | 2 | 0.68 | HLADRA | 2 | 0.67 |
| CCR2 | 2 | 0.69 | JAK1 | 2 | 0.67 |
| HLADRA | 2 | 0.7 | IFI16 | 2 | 0.69 |
| MNDA | 2 | 0.7 | IL32 | 2 | 0.73 |
| IL5 | 2 | 0.8 | CYP3A4 | 2 | 0.74 |
| CD80 | 2 | 0.8 | CCL3 | 2 | 0.76 |
| STAT1 | 2 | 0.84 | STAT1 | 2 | 0.78 |
| IRF1 | 2 | 0.84 | IL23A | 2 | 0.83 |
| MHC2TA | 2 | 0.86 | PRF1 | 2 | 0.85 |
| CCL3 | 2 | 0.93 | MHC2TA | 2 | 0.89 |
| IFI16 | 2 | 0.98 | IL5 | 2 | 0.95 |

TABLE 4
R-squared = 0.82

| 2-gene | | | 2-gene | | |
|---|---|---|---|---|---|
| model | | | model | | |
| week 4 | | p-value | week 6 | | p-value |
| ICOS | 1 | 3.60E−10 | ICOS | 1 | 2.40E−06 |
| CD69 | 2 | 0.00028 | CD69 | 2 | 1.10E−07 |
| MHC2TA | 2 | 0.0018 | TNFRSF6 | 2 | 0.00075 |
| PLA2G7 | 2 | 0.0038 | PLA2G7 | 2 | 0.0017 |
| TOSO | 2 | 0.0057 | MHC2TA | 2 | 0.0027 |
| PRF1 | 2 | 0.011 | TOSO | 2 | 0.0028 |
| GZMB | 2 | 0.013 | PLAU | 2 | 0.003 |
| IL1R1 | 2 | 0.014 | TNFRSF5 | 2 | 0.0032 |
| CCL3 | 2 | 0.016 | JUN | 2 | 0.0064 |
| SSI3 | 2 | 0.018 | HLADRA | 2 | 0.02 |
| THBS1 | 2 | 0.028 | SSI3 | 2 | 0.021 |
| IL12B | 2 | 0.04 | CDKN1A | 2 | 0.034 |
| TNFRSF5 | 2 | 0.047 | ADAM17 | 2 | 0.036 |
| JUN | 2 | 0.06 | CCR2 | 2 | 0.039 |
| IL32 | 2 | 0.081 | IL1R1 | 2 | 0.041 |
| HLADRA | 2 | 0.17 | THBS1 | 2 | 0.051 |
| JAK1 | 2 | 0.18 | PRF1 | 2 | 0.059 |
| CCR5 | 2 | 0.19 | STAT1 | 2 | 0.1 |
| TNFRSF6 | 2 | 0.2 | GZMB | 2 | 0.11 |
| PLAU | 2 | 0.2 | CCL3 | 2 | 0.14 |
| PTGS2 | 2 | 0.22 | JAK1 | 2 | 0.15 |
| IL5 | 2 | 0.23 | IL12B | 2 | 0.18 |
| TNFSF10 | 2 | 0.25 | IRF5 | 2 | 0.24 |
| IL1RN | 2 | 0.26 | RAF1 | 2 | 0.28 |
| IL23A | 2 | 0.26 | CCR5 | 2 | 0.29 |
| CYP3A4 | 2 | 0.28 | CYP3A4 | 2 | 0.31 |
| ADAM17 | 2 | 0.35 | TNFSF10 | 2 | 0.34 |
| TSC22D3 | 2 | 0.43 | IL2RA | 2 | 0.41 |
| STAT1 | 2 | 0.48 | IRF1 | 2 | 0.54 |
| RAF1 | 2 | 0.49 | IFI16 | 2 | 0.55 |
| DUSP6 | 2 | 0.49 | PTGS2 | 2 | 0.57 |
| CCR2 | 2 | 0.56 | PLAUR | 2 | 0.6 |
| IRF1 | 2 | 0.56 | IL5 | 2 | 0.61 |
| CDKN1A | 2 | 0.61 | DUSP6 | 2 | 0.65 |
| CD80 | 2 | 0.68 | IL32 | 2 | 0.68 |
| IRF5 | 2 | 0.69 | LTA | 2 | 0.75 |
| LTA | 2 | 0.69 | IL1RN | 2 | 0.8 |
| IFI16 | 2 | 0.83 | CD80 | 2 | 0.81 |
| TIMP1 | 2 | 0.85 | SERPINA1 | 2 | 0.88 |
| MNDA | 2 | 0.89 | IL23A | 2 | 0.9 |
| SERPINA1 | 2 | 0.9 | MNDA | 2 | 0.93 |
| IL2RA | 2 | 0.92 | CCL1 | 2 | 0.96 |
| CCL1 | 2 | 0.95 | TIMP1 | 2 | 0.99 |
| PLAUR | 2 | 0.98 | TSC22D3 | 2 | 0.99 |

TABLE 5
R-squared = 0.72

| 2-gene | | | 2-gene | | |
|---|---|---|---|---|---|
| model | | | model | | |
| week 4 | | p-value | week 6 | | p-value |
| IL32 | 1 | 3.60E−09 | IL32 | 1 | 6.40E−05 |
| CD69 | 2 | 0.00011 | CD69 | 2 | 4.40E−07 |
| TOSO | 2 | 0.0022 | TNFRSF6 | 2 | 0.00011 |
| IL1R1 | 2 | 0.0032 | TOSO | 2 | 0.00017 |
| PLA2G7 | 2 | 0.0043 | CDKN1A | 2 | 0.0016 |
| TSC22D3 | 2 | 0.0057 | TNFRSF5 | 2 | 0.002 |
| ICOS | 2 | 0.0061 | PLA2G7 | 2 | 0.0022 |
| LTA | 2 | 0.0069 | PLAU | 2 | 0.0024 |
| IL23A | 2 | 0.0089 | ADAM17 | 2 | 0.0041 |
| CDKN1A | 2 | 0.012 | JUN | 2 | 0.0057 |
| TNFRSF6 | 2 | 0.022 | IL1R1 | 2 | 0.011 |
| DUSP6 | 2 | 0.026 | ICOS | 2 | 0.011 |
| MHC2TA | 2 | 0.043 | SSI3 | 2 | 0.019 |
| SSI3 | 2 | 0.052 | MHC2TA | 2 | 0.027 |
| TNFRSF5 | 2 | 0.061 | HLADRA | 2 | 0.033 |
| ADAM17 | 2 | 0.077 | CCR2 | 2 | 0.064 |
| JAK1 | 2 | 0.091 | IL23A | 2 | 0.065 |
| CCL3 | 2 | 0.11 | RAF1 | 2 | 0.13 |
| JUN | 2 | 0.11 | DUSP6 | 2 | 0.17 |
| TNFSF10 | 2 | 0.13 | JAK1 | 2 | 0.17 |
| GZMB | 2 | 0.21 | TSC22D3 | 2 | 0.18 |
| RAF1 | 2 | 0.23 | CCL3 | 2 | 0.22 |
| HLADRA | 2 | 0.31 | IL12B | 2 | 0.23 |
| PRF1 | 2 | 0.32 | LTA | 2 | 0.23 |
| IL1RN | 2 | 0.4 | TNFSF10 | 2 | 0.24 |
| IL5 | 2 | 0.43 | PLAUR | 2 | 0.28 |
| IL12B | 2 | 0.48 | PTGS2 | 2 | 0.34 |
| PLAU | 2 | 0.48 | GZMB | 2 | 0.35 |
| TIMP1 | 2 | 0.5 | PRF1 | 2 | 0.36 |
| PTGS2 | 2 | 0.52 | CYP3A4 | 2 | 0.37 |
| MNDA | 2 | 0.53 | IRF5 | 2 | 0.39 |
| IL2RA | 2 | 0.55 | TIMP1 | 2 | 0.46 |
| PLAUR | 2 | 0.58 | IL2RA | 2 | 0.54 |
| CD80 | 2 | 0.63 | STAT1 | 2 | 0.57 |
| IRF1 | 2 | 0.68 | IRF1 | 2 | 0.57 |
| THBS1 | 2 | 0.68 | SERPINA1 | 2 | 0.61 |
| CCR2 | 2 | 0.7 | CCR5 | 2 | 0.63 |
| IRF5 | 2 | 0.74 | CCL1 | 2 | 0.63 |
| SERPINA1 | 2 | 0.77 | MNDA | 2 | 0.74 |
| CCR5 | 2 | 0.78 | IFI16 | 2 | 0.74 |
| CYP3A4 | 2 | 0.8 | IL1RN | 2 | 0.78 |
| IFI16 | 2 | 0.81 | THBS1 | 2 | 0.82 |
| STAT1 | 2 | 0.85 | CD80 | 2 | 0.82 |
| CCL1 | 2 | 0.87 | IL5 | 2 | 0.87 |

TABLE 6
R-squared = 0.55 0.55

| 2-gene | | | 2-gene | | |
|---|---|---|---|---|---|
| model | | | model | | |
| week 4 | | p-value | week 6 | | p-value |
| LTA | 1 | 6.10E−08 | LTA | 1 | 0.00039 |
| IL1R1 | 2 | 1.70E−06 | TOSO | 2 | 2.20E−05 |
| SSI3 | 2 | 5.20E−05 | TNFRSF6 | 2 | 4.50E−05 |
| TOSO | 2 | 8.60E−05 | CD69 | 2 | 7.50E−05 |
| IL1RN | 2 | 0.00012 | PLAU | 2 | 0.00016 |
| IL32 | 2 | 0.00027 | IL1R1 | 2 | 0.00025 |
| ICOS | 2 | 0.00034 | JUN | 2 | 0.00034 |
| PRF1 | 2 | 0.00042 | SSI3 | 2 | 0.0012 |
| TNFSF10 | 2 | 0.00075 | ADAM17 | 2 | 0.0014 |
| PLAU | 2 | 0.00082 | PLA2G7 | 2 | 0.0015 |
| IL23A | 2 | 0.0015 | TNFRSF5 | 2 | 0.0017 |
| CCR5 | 2 | 0.003 | ICOS | 2 | 0.0019 |
| MHC2TA | 2 | 0.0057 | MHC2TA | 2 | 0.0056 |
| GZMB | 2 | 0.0076 | CDKN1A | 2 | 0.0061 |
| TSC22D3 | 2 | 0.0097 | HLADRA | 2 | 0.0079 |
| RAF1 | 2 | 0.017 | IL23A | 2 | 0.018 |
| CCL3 | 2 | 0.018 | RAF1 | 2 | 0.019 |
| TNFRSF6 | 2 | 0.021 | TNFSF10 | 2 | 0.019 |
| SERPINA1 | 2 | 0.021 | IL32 | 2 | 0.029 |
| HLADRA | 2 | 0.04 | PRF1 | 2 | 0.033 |
| IL12B | 2 | 0.04 | CCR5 | 2 | 0.041 |
| CD69 | 2 | 0.048 | CCR2 | 2 | 0.042 |
| TNFRSF5 | 2 | 0.062 | GZMB | 2 | 0.055 |
| PLA2G7 | 2 | 0.069 | PTGS2 | 2 | 0.062 |
| MNDA | 2 | 0.087 | SERPINA1 | 2 | 0.088 |
| ADAM17 | 2 | 0.1 | IL1RN | 2 | 0.094 |
| PLAUR | 2 | 0.11 | PLAUR | 2 | 0.095 |
| IFI16 | 2 | 0.16 | CCL3 | 2 | 0.15 |
| IL5 | 2 | 0.16 | IL12B | 2 | 0.17 |
| JUN | 2 | 0.16 | DUSP6 | 2 | 0.19 |
| CD80 | 2 | 0.16 | TSC22D3 | 2 | 0.3 |
| DUSP6 | 2 | 0.19 | MNDA | 2 | 0.36 |
| CDKN1A | 2 | 0.21 | IL5 | 2 | 0.36 |
| TIMP1 | 2 | 0.24 | JAK1 | 2 | 0.37 |
| PTGS2 | 2 | 0.43 | THBS1 | 2 | 0.39 |
| IL2RA | 2 | 0.47 | TIMP1 | 2 | 0.41 |
| JAK1 | 2 | 0.53 | IFI16 | 2 | 0.43 |
| CCL1 | 2 | 0.64 | IRF5 | 2 | 0.45 |
| CCR2 | 2 | 0.74 | STAT1 | 2 | 0.56 |
| CYP3A4 | 2 | 0.76 | CYP3A4 | 2 | 0.57 |
| IRF1 | 2 | 0.78 | CD80 | 2 | 0.61 |
| STAT1 | 2 | 0.85 | IRF1 | 2 | 0.63 |
| IRF5 | 2 | 0.89 | IL2RA | 2 | 0.73 |
| THBS1 | 2 | 0.92 | CCL1 | 2 | 0.87 |

What is claimed is:

1. A method for determining a profile data set for characterizing a subject with transplant rejection or an inflammatory condition related to transplant rejection based on a sample from the subject, the sample providing a source of RNAs, the method comprising:
using amplification for measuring the amount of RNA in a panel of constituents including at least 1 constituent from any of Tables 1, 2 3, 4, 5, or 6 and
arriving at a measure of the constituent;
wherein the profile data set comprises the measure of each constituent of the panel and wherein amplification is performed under measurement conditions that are substantially repeatable.

2. A method of characterizing transplant rejection or an inflammatory condition related to transplant rejection in a subject, based on a sample from the subject, the sample providing a source of RNAs, the method comprising:
assessing a profile data set of a plurality of members, each member being a quantitative measure of the amount of a distinct RNA constituent in a panel of constituents selected so that measurement of the constituents enables characterization of the presumptive signs of a transplant rejection, wherein such measure for each constituent is obtained under measurement conditions that are substantially repeatable.

3. The method claim 1, wherein the panel comprises 10 or fewer constituents.

4. The method of claim 1, wherein the panel comprises 5 or fewer constituents.

5. The method of claim 1, wherein the panel comprises 2 constituents,

6. A method of characterizing according to claim 1, wherein the panel of constituents is selected so as to distinguish from a normal subject and a subject that will reject a transplant.

7. The method of claim 6, wherein the panel of constituents distinguishes from a normal subject and a subject rejecting a transplant with at least 75% accuracy.

8. The method of claim 1, wherein the panel of constituents is selected as to permit characterizing severity of transplant reject in relation to normal over time so as to track movement toward normal as a result of successful therapy and away from normal in response to transplant rejection.

9. The method of claim 1, wherein the panel includes TOCO, ICOS, IL31 or LTA.

10. A method according to claim 9, wherein the panel further includes CD69, or IL1R1

11. The method of claim 2, wherein the panel includes two or more constituents from Table 1.

12. A method of characterizing transplant rejection or an inflammatory condition related to transplant rejection in a subject, based on a sample from the subject, the sample providing a source of RNAs, the method comprising:
determining a quantitative measure of the amount of at least one a constituent of Table 1 as a distinct RNA constituent, wherein such measure is obtained under measurement conditions that are substantially repeatable.

13. The method of claim 12, wherein said constituent is TOSO, IL32, or LTA.

14. The method of claim 13, further comprising determining a quantitative measure of at least one constituent selected from the group consisting of CD69 or IL1R1.

15. The method of claim 12, wherein the constituents distinguish from a normal and a transplant recipient with at least 75% accuracy.

16. A method of assessing the efficacy of a compound to suppress the immune system in a subject, based on a sample from the subject, the sample providing a source of RNAs, the method comprising:
contacting a first sample from said subject with a test compound and determining a first quantitative measure of the amount of at least one constituent from Table 1 or Table 2 in said first sample as a distinct RNA constituent to produce a test data set, wherein such measure is obtained under measurement conditions that are substantially repeatable; and
comparing the test data set to a baseline data set.

17. The method of claim 16, wherein said baseline data set is derived from a second sample from said subject.

18. The method of claim 17, wherein said second sample has not been exposed to said test compound.

19. A method of assessing the efficacy of a compound to suppress the immune system in a subject, based on a sample from the subject, the sample providing a source of RNAs, the method comprising:
determining a first quantitative measure of the amount of at least one constituent from Table 1 or Table 2 in a first sample from said subject that has been exposed to said compound as a distinct RNA constituent to produce a test data set, wherein such measure is obtained under measurement conditions that are substantially repeatable; and
comparing the test data set to a baseline data set.

20. The method of claim 19, wherein said baseline data set is derived from a second sample from said subject.

21. The method of claim 20, wherein said second sample has not been exposed to said compound.

22. The method of claim 20, wherein said second sample is obtained from said subject prior to exposure to said compound.

23. The method of claim 20, wherein said second sample is obtained from said subject after exposure to said compound

24. A method for determining a profile data set according to claim 1, wherein the measurement conditions that are substantially repeatable are within a degree of repeatability of better than five percent.

25. The method of claim 1, wherein the measurement conditions that are substantially repeatable are within a degree of repeatability of better than three percent.

26. The method of claim 1, wherein efficiencies of amplification for all constituents are substantially similar.

27. The method of claim 1, wherein the efficiency of amplification for all constituents is within two percent.

28. The method of claim 1, wherein the efficiency of amplification for all constituents is less than one percent.

29. The method of claim 1, wherein the sample is selected from the group consisting of blood, a blood fraction, bodily fluid, a population of cells and tissue from the subject.

30. The method of claim 2, wherein assessing further comprises:
comparing the profile data set to a baseline profile data set for the panel, wherein the baseline profile data set is related to transplant rejection or inflammatory conditions related to transplant rejection.
